(19) United States
(12) Patent Application Publication
(10) Pub. No.: US 2014/0171510 A1
(43) Pub. Date: Jun. 19, 2014

(54) HOMOARGININE PRODRUGS AND/OR CONJUGATES OF AMPHETAMINE AND OTHER STIMULANTS AND PROCESSES FOR MAKING AND USING THE SAME

(71) Applicant: Travis Mickle, Coralville, IA (US)

(72) Inventor: Travis Mickle, Coralville, IA (US)

(73) Assignee: Shire LLC, Florence (KY)

(21) Appl. No.: 14/235,743

(22) PCT Filed: Jul. 29, 2011
(86) PCT No.: PCT/US2011/045954

§ 371 Date: Feb. 4, 2014

(51) Int. Cl. A61K 47/48

(52) U.S. Cl. CPC A61K 47/48038
USPC 514/626; 564/196

(57) ABSTRACT

Disclosed are homoarginine amphetamine prodrug and/or conjugate compositions, salts thereof, or a combination thereof that can reduce or prevent amphetamine side effects in a human subject, and methods to reduce or prevent amphetamine side effects in a human subject.

### BACKGROUND OF THE INVENTION

The present technology relates to an improved dosage form of a homoarginine amphetamine prodrug and/or conjugate that reduces or prevents amphetamine side effects in a human subject. Additionally, the presently described technology also relates generally to methods of reducing or preventing amphetamine side effects in a human.

Stimulants, including amphetamine and its derivatives, enhance the activity of the sympathetic nervous system and/or central nervous system (CNS) and are prescribed for the treatment of a range of conditions and disorders predominantly encompassing, for example, attention deficit hyperactivity disorder (ADHD), attention deficit disorder (ADD), obesity, narcolepsy, appetite suppression, depression, anxiety and wakefulness.

Attention deficit hyperactivity disorder (ADHD) in children has been treated with stimulants for many years. However, more recently, the increase in the number of prescriptions for ADHD therapy in an adult population has, at times, outperformed the growth of the pediatric market. Although there are various drugs currently in use for the treatment of ADHD, such as methylphenidate (commercially available from, for example, Novartis International AG (located in Basel, Switzerland) under the trademark Ritalin®) and non-stimulant atomoxetine (commercially from Eli Lilly and Company (located in Indianapolis, Ind.) as Strattera®), amphetamine has been the forerunner in ADHD therapy. Moreover during classroom trials, non-stimulants have been shown to be less effective in improving behavior and attention of ADHD afflicted children than amphetamine derivatives.

Initial drug therapy for ADHD was limited to fast acting immediate release formulations of stimulants (e.g., Dexedrine®, pure dextroamphetamine sulfate, commercially available from Smith Kline and French located in the United Kingdom) which triggered an array of potentially undesirable side effects including, for example, fast wear-off of the therapeutic effect of the stimulant active ingredient causing rebound symptoms, cardiovascular stress/disorders (e.g., increased heart rate, hypertension, cardiomyopathy), other side effects (e.g., insomnia, euphoria, psychotic episodes), addiction and abuse.

Behavioral deterioration (rebound/“crashing”) is observed in a significant portion of children with ADHD as the medication wears off, typically in the afternoon or early evening. Rebound symptoms include, for example, irritability, crankiness, hyperactivity worse than in the unmedicated state, sadness, crying and in rare cases psychotic episodes. The symptoms may subside quickly or last several hours. Some patients may experience rebound/crashing so severe that treatment must be discontinued. Rebound/crashing effects can also give rise to addictive behavior by enticing patients to administer additional doses of stimulant with the intent to prevent anticipated rebound/crashing negative outcomes and side effects.

Stimulants, such as methylphenidate and amphetamine, have been shown to exhibit noradrenergic and dopaminergic effects that can lead to cardiovascular events comprising, for example, increased heart rate, hypertension, palpitations, tachycardia and in isolated cases cardiomyopathy, stroke, myocardial infarction and sudden death. Consequently, currently available stimulants expose patients with pre-existing structural cardiac abnormalities or other severe cardiac indications to even greater health risks and are frequently not used or used with caution in this population. It is notable, however that the cardiovascular effects of stimulants, for example, on heart rate and blood pressure is dependent on the administered dose. As a result, a treatment which maintains the lowest effective stimulant blood concentrations for a therapeutically beneficial duration is believed to demonstrate fewer cardiovascular risks/side effects.

Amphetamine and many of its derivatives (e.g., methamphetamine, 3,4-methylenedioxy-methamphetamine/“Ecstasy”) are widely abused for various purposes such as euphoria, extended periods of alertness/wakefulness, or rapid weight loss or by actual ADHD patients who developed excessive self-dosing habits to prevent rebound symptoms from manifesting, for example, in anxiety or depression. The effects desired by potential abusers originated from the stimulation of the central nervous system and prompted a Schedule II or even Schedule I classification for amphetamine (d- and l-amphetamine individually and any combination of both are Schedule II) and certain derivatives thereof after passage of the Controlled Substance Act (CSA) in 1970. Both classifications are defined by the high propensity for abuse. Schedule II drugs have an accepted medical use while Schedule I substances do not pursuant to the CSA. So far, all amphetamine products, including compositions with sustained release formulations and prodrugs thereof, are obligated to include a black box warning on the drug label to inform patients about the potential for amphetamine abuse and dependence.

It has been observed in the conventional art that most side effects of amphetamines are caused by a large initial spike in blood concentration of the stimulant which quickly erodes to levels below therapeutic effectiveness (typically within 4-6 hours). As a consequence, the high potency of dextroamphetamine (d-amphetamine) was subsequently modulated by a series of new drugs with increasingly sustained release profiles achieved by delivering amphetamine more slowly into the blood stream with the goal to create safer and less abusable treatment outcomes and regimens. The methods and technologies for generating smaller spikes in drug blood concentrations include, for example, use of mixed salts and isomer compositions (i.e., different salts of d- and less potent 1-amphetamine), extended/controlled/sustained release formulations (e.g., Adderall X® commercially available from Shire U.S., Inc. located in Wayne, Pa.) and, most recently, prodrugs of amphetamine (Vyvanse™ also commercially available from Shire). The ideal drug treatment option should produce stimulant blood concentrations within a narrow therapeutic window for an extended time duration followed by a prolonged fade-out period in order to minimize cardiovascular stress and behavioral deterioration, and would also exhibit anti-abuse properties.

Besides immediate release formulations, newer sustained release formulations have been developed with the objective to provide a therapeutic treatment option that offers the convenience of a single daily dosing regimen versus multiple quotidian administrations. Such formulations also have the objective of imparting or rendering a euphoric response. Sustained release formulations commonly consist of drug particles coated with a polymer or polymer blend that delays and extends the absorption of the active drug substance by the gastrointestinal tract for a relatively defined period of time. Such formulations frequently embed the therapeutic agent/active ingredient/drug within a hydrophilic hydrocolloid gelling polymer matrix (e.g., hydroxypropyl methylcellulose, hydroxypropyl cellulose or pullulan). This dosage formulation in turn becomes a gel upon entering an acidic medium, as found in the stomach of humans and animals, thereupon slowly effusing the therapeutic agent/active ingredient/drug. However, the dosage formulation dissolves in an alkaline medium, as found in the intestines of humans and animals, concurrently liberating the drug more quickly in an uncontrolled manner. Some formulations, such as acrylic resins, acrylic latex dispersions, cellulose acetate phthalate, and hydroxypropyl methylcellulose phthalate, offer improved sustained release in the intestines by being resistant to acidic environments and dispensing the active ingredient only at elevated pH via a diffusion-erosion mechanism, either by themselves or mixed with hydrophilic polymers.

Sustained release formulations have been moderately effective in providing an improved and extended dosage form over immediate release tablets. Nonetheless, such formulations are potentially subject to inconsistent, erratic or premature release of the therapeutic agent due to failure of the polymer material, and they also usually allow easy extraction of the active ingredient utilizing a simple physical procedure. Since single daily dose formulations contain a greater amount of amphetamine than immediate release formulations, they are more attractive to potential abusers, consequently making the extractability of drug substance an additional undesirable property. It is also, at least in part, a reason for increased drug diversion, especially evident by selling or trading of medication by school children who are ADHD patients and in possession of sustained release amphetamine capsules. The obtained stimulants are then abused by classmates without the disorder by either ingesting high doses or snorting the drug material after crushing it.

U.S. Pat. No. 7,105,486 (to assignee New River Pharmaceuticals, hereinafter the “486 patent”) appears to describe compounds comprising a chemical moiety (namely L-lysine) covalently attached to amphetamine, compositions thereof, and methods of using the same. Allegedly, these compounds and their compositions are useful for reducing or preventing abuse and overdose of amphetamine. The '486 patent also describes that using any amino acid other than l-lysine (Table 46) will not give rise to the same in vivo properties demonstrated by l-lysine-d-amphetamine (Lys-Amp, Vyvanse™). In humans, at least some of the standard amino acid conjugate is absorbed and enters the circulatory system as the intact conjugate rather than the cleaved amphetamine. The presence of the intact conjugate in the bloodstream can lead to unforeseen side effects. As a result, there still exists a need within the art for a safer dosage form of amphetamine, and patient compliant treatment regimen that is therapeutically effective, can provide sustained release and sustained therapeutic effect, and can limit the side effects that can occur due to the presence of the intact amphetamine conjugate in the blood stream ([a] Boellner, S. W.; et al. Pharmacokinetics of lisdexamfetamine dimesylate and its active metabolite, d-amphetamine, with increasing oral doses of lisdexamfetamine dimesylate in children with attention-deficit/hyperactivity disorder: a single-dose, randomized, open-label, crossover study. Clinical Therapeutics 2010, 32(2), 252-264. [b] Krishnan, S. M.; et al. Metabolism, distribution and elimination of lisdexamfetamine dimesylate: open-label, single-centre, phase I study in healthy adult volunteers. Clinical Drug Investigation 2008, 28(12), 745-755. [c] Krishnan, S.; et al. Relative bioavailability of lisdexamfetamine 70-mg capsules in fasted and fed healthy adult volunteers and in solution: a single-dose, crossover pharmacokinetic study. Journal of Clinical Pharmacology 2008, 48(30), 293-302. [d] Krishnan, S. M.; et al. Multiple daily-dose pharmacokinetics of lisdexamfetamine dimesylate in healthy adult volunteers. Current Medical Research and Opinion 2008, 24(1), 33-40. [e] Emer, J.; et al. Lisdexamfetamine dimesylate: linear dose-proportionality, low intersubject and intrasubject variability, and safety in an open-label single-dose pharmacokinetic study in healthy adult volunteers. Journal of Clinical Pharmacology 2010, 50(9), 1001-1010).

### BRIEF SUMMARY OF THE INVENTION

In general, the presently described technology in at least one aspect is directed to an improved dosage form of homoarginine amphetamine conjugate/prodrug that not only allows slow/sustained/controlled delivery of the amphetamine into the blood system of a human within a safe therapeutic window upon oral administration, but also limits the amount of detectable prodrug in the bloodstream.

Thus, the presently described technology provides one or more compositions comprising at least one conjugate of homoarginine amphetamine, a salt thereof, or a combination thereof, which exhibits a plasma concentration in a human subject of the intact homoarginine amphetamine conjugate that is below the limit of quantitation. The amphetamine chemically attached to (preferably covalently attached to) the homoarginine nonstandard amino acid to form the conjugate is converted into its active form in the body by normal metabolic processes.

Although not wanting to be bound by any particular theory, the homoarginine amphetamine conjugate of the present technology is believed to be safer than other sustained release forms of amphetamine by providing controlled blood levels of amphetamine for a prolonged period of time, thus preventing the rebound effect, cardiovascular stress and euphoria associated with conventional stimulant treatment options. Further, because the intact homoarginine amphetamine conjugate is not absorbed into the bloodstream of a human, the homoarginine amphetamine conjugate is believed to reduce or prevent side effects that can occur with other amphetamine conjugates that enter the bloodstream as the intact amphetamine conjugate.

The presently described technology further provides methods of reducing or preventing amphetamine side effects by orally administering at least one homoarginine amphetamine conjugate to a human subject. Release of amphetamine following oral administration of the homoarginine amphetamine conjugates of the present technology can occur gradually over an extended period of time thereby eliminating unintended elevations (e.g., blood level concentration spikes) of drug levels in the bloodstream of a human patient. For example, after a single oral dose of 25 mg of homoarginine-amphetamine, it takes approximately three hours until the maximum plasma concentration of amphetamine is reached in humans. Thereafter, amphetamine is slowly eliminated over a period of about 45 hours. Again not wanting to be bound by any particular theory, it is also believed that such spikes in blood levels can lead to euphoric drug “high” and/or cardiovascular effects like increased blood pressure and heart rate. Additionally, sustained blood levels are achieved within an effective therapeutic range for a longer duration than other conventional therapies, thereby preventing a rebound effect. Moreover, sustained blood levels of amphetamine are achieved as a result of the cleaved amphetamine, and not through absorption of the intact conjugate, thereby reducing the potential for side effects, toxicity and/or unknown or unwanted effects arising from the presence of the intact conjugate in the bloodstream.

The homoarginine amphetamine conjugates of the present technology preferably have no or a substantially decreased pharmacological activity when administered through injection or intranasal routes of administration. However, they remain orally bioavailable. Again, not wanting to be bound by any particular theory, the bioavailability can be a result of the hydrolysis of the chemical linkage (e.g., a covalent linkage) following oral administration. Hydrolysis of the chemical linkage is time-dependent, thereby allowing amphetamine to become available in its active form over an extended period of time, while limiting the availability of the intact conjugate. In at least one embodiment, release of amphetamine is diminished or eliminated when the composition of the present technology is delivered by parenteral routes.

In some embodiments of the present technology, the amphetamine can be a metabolite of amphetamine, a salt thereof, a derivative thereof, or a mixture thereof. Amphetamine can be in the form of dextro- (d-), levo- (l-), or racemic. One preferred amphetamine is d-amphetamine.

In another aspect, the presently described technology provides a method of reducing or preventing stimulant side effects in a human patient with a disorder or condition requiring the stimulation of the patient's CNS (Central Nervous System), comprising the step of orally administering to the patient in need, a composition formulated to comprise a dose of at least one homoarginine amphetamine, wherein the dose provides the equivalent of about 5 mg to about 40 mg of amphetamine freebase, and wherein the conjugate or salt thereof is below the limit of quantitation in the bloodstream of the human following the oral administration step. Alternatively, the dose may also be provided in an equivalent of about 9 mg to about 30 mg of amphetamine freebase.

In a further aspect, the presently described technology provides one or more compositions for reducing or preventing amphetamine or amphetamine derivative side effects in a human, the composition(s) comprising or including at least one orally administered homoarginine-amphetamine conjugate or salt thereof, or at least one homoarginine-amphetamine derivative conjugate or a salt thereof, wherein either of the conjugates or salts thereof are present in the composition in an amount equivalent to amphetamine freebase in the range of about 5 mg to about 40 mg, and wherein either of the conjugates or salts thereof are below the limit of quantitation in the bloodstream of the human following oral administration.

In at least one embodiment of the present technology, the chemical attachment (preferably covalent attachment) of the homoarginine nonstandard amino acid to the amphetamine in the composition can substantially decrease the potential for overdose when the composition is administered to the patient by decreasing the toxicity of the stimulant at doses above those considered therapeutic, while maintaining the active agent/ingredient's pharmaceutical activity within a normal dose range. Without being bound by any particular theory, it is believed that the homoarginine conjugated with amphetamine may decrease or eliminate the pharmacological activity of the amphetamine. Therefore, restoring activity requires release of the amphetamine from the homoarginine amphetamine conjugate.

Other objects, advantages and embodiments of the invention are described below and will be obvious from this description and practice of the invention.

### BRIEF DESCRIPTION OF SEVERAL VIEWS OF THE DRAWINGS

FIG. 1 compares mean plasma concentrations released from rats orally administered l-homoarginine-d-amphetamine or l-lysine-d-amphetamine.

FIG. 2 compares the relative blood levels of d-amphetamine released from 1-homoarginine-d-amphetamine and l-lysine-d-amphetamine.

FIGS. 3 and 4 illustrate the difference in blood levels obtained from the study results shown in FIG. 2.

FIG. 5 compares average plasma concentrations from four (4) oral studies of rats administered l-homoarginine-d-amphetamine or l-lysine-d-amphetamine.

FIG. 6 compares the mean plasma concentrations of d-amphetamine released from rats intranasally administered l-homoarginine-d-amphetamine or l-lysine-d-amphetamine.

FIG. 7 compares the mean plasma concentrations of d-amphetamine released from rats intravenously administered d-amphetamine, l-homoarginine-d-amphetamine or l-lysine-d-amphetamine.

FIG. 8 compares the PK curves of d-amphetamine released from rats orally administered homoarginine-d-amphetamine-2HCl and lysine-d-amphetamine.

FIG. 9 compares the PK curves of the intact prodrugs homoarginine-d-amphetamine-2HCl and lysine-d-amphetamine after oral administration to rats.

FIG. 10 compares the PK curves of d-amphetamine released from dogs orally administered homoarginine-d-amphetamine-2HCl and lysine-d-amphetamine.

FIG. 11 compares the PK curves of the intact prodrugs homoarginine-d-amphetamine-2HCl and lysine-d-amphetamine after oral administration to dogs.

FIG. 12 compares the PK curves of d-amphetamine released from human subjects orally administered homoarginine-d-amphetamine-2HCl and lysine-d-amphetamine.

FIG. 13 compares the PK curves of the intact prodrugs homoarginine-d-amphetamine-2HCl and lysine-d-amphetamine after oral administration to human subjects.

FIG. 14 compares the PK curves of d-amphetamine released from dogs orally administered l-homoarginine-d-amphetamine in solution and oral thin film dosage forms.

FIG. 15 compares the PK curves of the intact prodrug l-homoarginine-d-amphetamine after oral administration to dogs in solution and oral thin film dosage forms.

### DETAILED DESCRIPTION OF THE INVENTION

The presently described technology relates to one or more improved dosage forms of homoarginine amphetamine conjugates, salts thereof, or combinations thereof that reduce or prevent the side effects or other unwanted effects exhibited, observed, or potentially experienced from the presence of the intact amphetamine conjugate in the bloodstream. A preferred improved composition and/or dosage form is a homoarginine amphetamine dihydrochloride salt formulated into an oral thin strip. Methods of reducing or preventing amphetamine side effects in a human subject are also disclosed.

As used herein, a “non-standard” amino acid refers to a naturally occurring amino acid that is not one of the “standard” 20 amino acids. Non-standard amino acids do not have genetic codon, nor are they incorporated into proteins of natural origin. One category of non-standard amino acids are metabolites of other amino acids.

As used herein, “amphetamine” shall mean any of the sympathomimetic phenethylamine derivatives which have central nervous system stimulant activity, such as but not limited to, amphetamine (alpha-methyl-phenethylamine), methamphetamine, p-methoxyamphetamine, methylenedioxyamphetamine, 2,5-dimethoxy-4-methylamphetamine, 2,4,5-trimethoxyamphetamine, and 3,4-methylenedioxy-methamphetamine.

As used herein, “prodrug” shall mean a form of a drug that is not active on its own until it is metabolized in the body and made active.

As used herein, “in a manner inconsistent with the manufacturer's instructions” or similar expression is meant to include, but is not limited to, consuming amounts greater than amounts described on the label or ordered by a licensed physician, and/or altering by any means (e.g., crushing, breaking, melting, separating etc.) the dosage formulation such that the composition maybe injected, inhaled or smoked.

As used herein, the phrases such as “decreased,” “reduced,” “diminished” or “lowered” is meant to include at least a 10% change in pharmacological activity with greater percentage changes being preferred for reduction in abuse potential and overdose potential. For instance, the change may also be greater than 25%, 35%, 45%, 55%, 65%, 75%, 85%, 95%, 96%, 97%, 98%, 99%, or any and all increments therein.

As used herein, the phrase “substantially nondetectable in the bloodstream” refers to a concentration that is below the limit of quantitation of at least 1.00 ng/mL.

As used herein, the phrase “below the limit of quantitation” (LLOQ) generally refers to a concentration, below which the analysis of human fluid samples (e.g., plasma samples) regarding one or more selected active pharmaceutical ingredient(s) (“API(s)”, e.g., amphetamine) with a validated LC-MS/MS (liquid chromatography-mass spectrometry/tandem mass spectrometry) methodology and associated calibration curves and standards may not produce accurate data. In other words, if the analysis of a human fluid sample results in concentration values that are LLOQ, it can be accurately concluded that the concentration of the one or more selected active pharmaceutical ingredient(s) is not greater than the given LLOQ value. As an example, the administration of an oral solution containing at least a 25 mg dose of homoarginine amphetamine prodrug does not produce plasma concentrations of intact homoarginine amphetamine prodrug that exceed the LLOQ of 1.00 ng/mL.

A lower limit of quantitation (LLOQ) can be determined, for example, by the following method: Plasma samples are analyzed (three runs each) for amphetamine with a validated LC-MS/MS method. The calibration curve is prepared by plotting peak area ratios of amphetamine to the internal standard (amphetamine-d₅) against the concentrations of the plasma calibration standard. The calibration curve is then fitted by weight linear least squares regression analysis and found to be linear down to a concentration of 1.00 ng/mL (LLOQ). Precision and accuracy at the LLOQ are verified by analyzing six samples of the 1.00 ng/mL plasma standard and entering the observed peak areas into the derived equation for the least squares regression line to obtain “back-calculated” concentration values.

Some abbreviations that may be used in the present application include: DCC=dicyclohexylcarbodiimide, NHS=N-hydroxysuccinimide, EtOAc=ethyl acetate, MsOH=methanesulfonic acid, EDCI=1-Ethyl-3-(3-dimethylaminopropyl) carbodiimide, PyBrOP=Bromo-tris-pyrrolidino phosphoniumhexafluorophosphate, NMM=N-methylmorpholine or 4-methylmorpholine, TEA=triethylamine, CDI=Carbonyl diimidazole, IPAC=isopropyl acetate, DEA=diethylamine, BOP=(Benzotriazol-1-yloxy)tris(dimethylamino)phosphonium hexafluorophosphate.

According to the presently described technology, homoarginine can be chemically (preferably covalently) attached to amphetamine (d-, l-, or racemic form or a mixture thereof) to produce homoarginine prodrugs of amphetamine. Metabolites and derivatives of amphetamine could also be modified with the same potential benefit. Examples of metabolites of amphetamine include N-hydroxyamphetamine, 4-hydroxyamphetamine, α-hydroxyamphetamine, norephedrine, 4-hydroxynorephedrine, phenylacetone oxime, phenylacetone and 1-phenyl-2-propanol.

Salts of the homoarginine amphetamine prodrug that can be formed and utilized include, but are not limited to, mesylate, hydrochloride, sulfate, oxalate, triflate, citrate, malate, tartrate, phosphate, nitrate, benzoate, acetate, carbonate, hydroxide, sodium, potassium, magnesium, calcium, zinc, and ammonium salts. Further, in accordance with some embodiments, the salts may be required in multiple forms (e.g., di-, tri-, or tetra-). Other derivative forms such as free base, free acid, or neutral forms may also be prepared.

Generally, to conjugate homoarginine with amphetamine, the amino group and guanidino group are preferably protected before homoarginine is reacted with amphetamine. Agents and methods for protecting amino groups and guanidino groups in a reactant are known in the art. Examples of protecting groups that may be used to protect the amino groups include, but are not limited to, fluorenylmethoxycarbonyl (Fmoc), t-butylcarbonate (Boc), trifluoroacetate (TFA), and benzyloxycarbonyl (Z). Additional protection of the guanidino group may be necessary. Examples of protecting groups that may be used to protect the guanidino group include, but are not limited to, t-butylcarbonate (Boc), benzyloxycarbonyl (Z) and nitro. After coupling with any standard coupling procedure, deprotection can occur depending on protecting groups, for example, via catalytic hydrogenation using a catalyst such as palladium-carbon in the presence of hydrogen gas or any other hydride donor molecule, and/or with a variety of strong acids, such as hydrochloric acid, sulfuric acid, hydrobromic acid, or methanesulfonic acid, to give the corresponding salt form. Examples of other catalysts that could be used in place of palladium-carbon include titanium trichloride (TiCl₃) tin dichloride (5 nCl₂), Raney nickel, platinum (IV) oxide (PtO₂), samaribum diiodide (SmI₂), Ushibara catalysts (for example, U—Ni-A, U—Ni—B, U—Ni—BA, U—Ni-AA, U—Ni—NH₃, U—Co-A, U—Co—B, U—Fe(II); where A=acid, B-base, BA-base with aluminum, AA=acid with aluminum), and iron metal. Salt forms may also be switched by first free basing the product and then adding any acid. Neutral, free base or anionic salts may also be formed.

The amino acid whose amino group and guanidino group are protected can be referred to as an N-protected amino acid. One can either protect the amino groups before the coupling reaction or use commercially available N-protected amino acids directly. Preferably, the carboxylic acid group in the N-protected amino acid is activated by an acid activating agent (sometimes also called coupling reagent) to help the reaction of the N-protected amino acid with amphetamine. General information about the reaction of amino acids to form peptide bonds can be found in, for example, G. C. Barett, D. T. Elmare, Amino Acids and Peptides, page 151-156, Cambridge University Press, UK (1st edition, 1998); Jones, J., Amino Acid and Peptide Synthesis, pages 25-41, Oxford University Press, UK (2nd edition, 2002), which are incorporated herein by reference in their entirety.

One category of acid activating agents (coupling reagents) well known in the art are carbodiimides. Examples of carbodiimide acid activating agents include, but are not limited to, dicyclohexylcarbodiimide (DCC), 1-ethyl-3-(3′-dimethylaminopropyl)-carbodiimide (EDCI), and diisopropylcarbodiimide (DIPCDI). Examples of other coupling reagents that could be used include bromo-tris-pyrrolidino phosphoniumhexafluorophosphate, (benzotriazol-1-yloxy)-tris-(dimethylamino)-phosphonium hexafluorophosphate, PCl₅/PhH, SOCl₂, N₂H₄, 1-ethoxycarbonyl-2-ethoxy-1,2-dihydroquinoline, other phosphonium reagents, and uronium reagents. The use of appropriate acyl halide or anhydride is also contemplated.

Depending on the protecting groups, the N-protected amino acid conjugate of amphetamine resulting from the reaction of the N-protected amino acid and amphetamine as described above can then be de- or un-protected in one step, for example via hydrogenation, or in two steps, for example via hydrogenation followed by treatment with a strong acid such as hydrochloric acid, hydrobromic acid, sulfuric acid or methanesulfonic acid to produce the corresponding final salt form of the amino acid conjugate of amphetamine.

Scheme 1 below outlines an exemplary route for the synthesis of a derivative of amphetamine chemically attached to homoarginine in accordance with the presently described technology. In this exemplary reaction scheme, an HCl salt form of homoarginine-amphetamine is produced. The procedure uses tert-butyloxycarbonyl (Boc) and nitro protected homoarginine (Boc-homoarginine(Nitro)) as the starting material. In this exemplary reaction scheme, coupling agent EDCI is added to Boc-homoarginine. N-hydroxy succinamide (NHS) is then added to the reaction mixture in dimethylformamide (DMF). A stable, yet still activated, succinic ester of Boc-homoarginine(nitro) is formed. Amphetamine is then added to the resulting succinic ester of Boc-homoarginine(nitro) to make the corresponding protected prodrug, Boc-homoarginine(nitro)-Amp. This protected prodrug can be de- or un-protected using hydrogenation followed by a strong acid such as methanesulfonic acid (MsOH) or hydrochloric acid to produce the prodrug of amphetamine, which is a hydrochloride salt of homoarginine-amphetamine in the exemplary reaction Scheme 1 and Scheme 2.

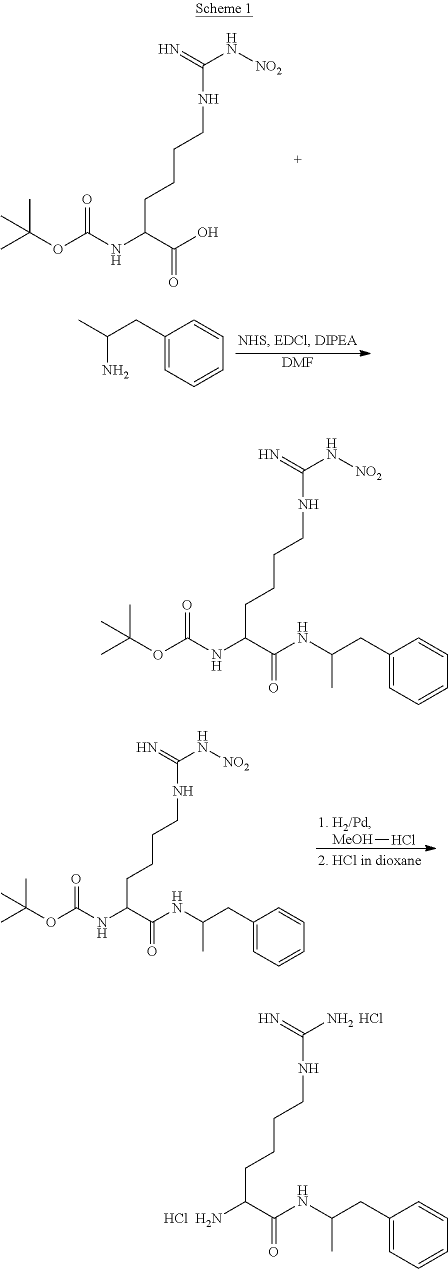

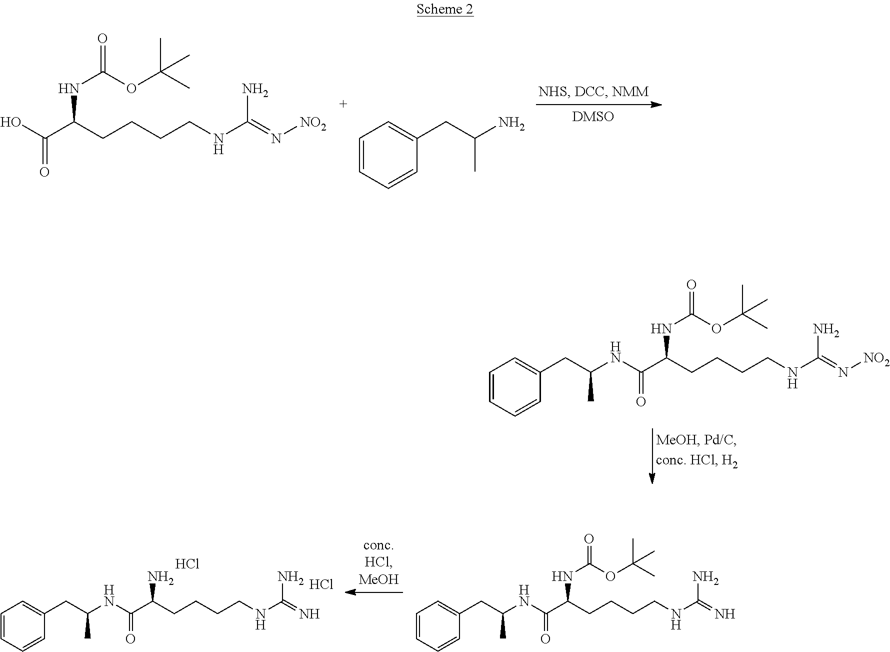

Alternative reaction conditions that may deprotect the nitro group (with or without catalyst and/or hydrogen) include palladium-carbon catalyst with cyclohexadiene, palladium-carbon catalyst with formic acid and methanol, titanium chloride at a pH of 6, tin dichloride with formic acid, electrolysis with 1N sulfuric acid, and oxygen gas in the presence of water and acid.

Examples of other solvents that can be used in the presently described technology include, but are not limited to, isopropyl acetate (IPAC), acetone, and dichloromethane (DCM), dimethylformamide (DMF), 2-methyltetrahydrofuran (2-MeTHF), ethyl acetate, chloroform, dimethyl sulfoxide, dioxane, diethyl ether, methyl t-butyl ether, hexanes, heptane, methanol, ethanol, isopropanol, and butanol. A mixture of different solvents can also be used. Co-bases such as tertiary amines may or may not be added in the coupling reaction of the presently described technology. Examples of suitable co-bases include, but are not limited to, 1-methylmorpholine (NMM), 4-methylmorpholine, triethylamine (TEA), ammonia or any tertiary amine base.

In a preferred embodiment, the homoarginine amphetamine prodrug comprises a dihydrochloride salt of homoarginine amphetamine. The pH of this prodrug in deionized water at various concentrations is as follows:

Concentration

| [mg/mL] | [mM] | pH |
|---|---|---|
| 0.1 | 0.3 | 6.15 ± 0.1 |
| 1.0 | 2.6 | 5.40 ± 0.1 |
| 10 | 26.4 | 4.35 ± 0.1 |
| 100 | 264.3 | 3.30 ± 0.1 |

The amphetamine to be chemically attached to homoarginine can be in d-form, l-form, or racemic form, or can be a mixture thereof. In accordance with some embodiments of the presently described technology, d-amphetamine (dextroamphetamine) and homoarginine are used to make an amphetamine prodrug. In accordance with some other embodiments, the prodrugs of d-amphetamine can be used in combination with a prodrug of l-amphetamine or l-amphetamine itself.

The homoarginine amphetamine prodrugs of the present technology have no or a substantially decreased pharmacological activity when administered through injection or intranasal routes of administration. However, they remain orally bioavailable. The bioavailability is the result of the hydrolysis of the covalent linkage following oral administration. Hydrolysis of a chemical linkage is time-dependent, thereby allowing amphetamine and other metabolites such as 4-hydroxyamphetamine and 4-hydroxynorephedrine to become available in their active form over an extended period of time. Therefore, the prodrug compounds of the present technology can release amphetamine or another stimulant over an extended period and provide a therapeutically area under the curve (AUC) when compared to free amphetamine, with little or no spike in concentration max (Cₘₐₓ)) or equivalent Cₘₐₓ. Not wanting to be bound by any particular theory, it is believed that since homoarginine is used to produce the prodrugs, the in vivo breakdown of the prodrugs by enzymes would occur at a slower rate leading to lower exposure of d-amphetamine. This will allow this type of prodrug to release amphetamine slowly and, preferably, only under in vivo conditions. Another theory could be that intact homoarginine-amphetamine is absorbed poorly or slowly in the gut due to, for example, the very polar amino acid side chain, not being recognized by amino acid transporters or being a substrate for intestinal efflux transporters. If the release of amphetamine, for example, occurs in the gut wall and the homoarginine-amphetamine conjugate is repeatedly taken up and pumped out of the enterocytes, the intact prodrug is only exposed to hydrolytic enzymes for short periods at a time. Consequently, a significant portion of the administered dose of homoarginine-amphetamine would have to be subjected to numerous iterations of absorption and efflux to release amphetamine resulting in an extended and attenuated release profile.

As a person of ordinary skill in the art will understand, drug products are considered pharmaceutical equivalents if they contain the same active ingredient(s), are of the same dosage form, route of administration and are identical in strength or concentration. Pharmaceutically equivalent drug products are formulated to contain the same amount of active ingredient in the same dosage form and to meet the same or compendial or other applicable standards (i.e., strength, quality, purity, and identity), but they may differ in characteristics such as shape, scoring configuration, release mechanisms, packaging, excipients (including colors, flavors, preservatives), expiration time, and, with certain limits, labeling. Drug products are considered to be therapeutic equivalents only if they are pharmaceutical equivalents and if they can be expected to have the same clinical effect and safety profile when administered to patients under the conditions specified in the labeling. The term “bioequivalent,” on the other hand, describes pharmaceutical equivalent or pharmaceutical alternative products that display comparable bioavailability when studied under similar experimental conditions.

Once produced, the prodrug of amphetamine (or another stimulant) of the present technology can be administered through oral routes of delivery and once administered will release the stimulant under digestive conditions. Not wanting to be bound by any particular theory, it is likely that any intact homoarginine-amphetamine is absorbed farther down the gastrointestinal tract, for example in the lower intestines, where the pH is higher and more prodrug is present in the non-ionized form. Due to the hydrophilic and polar nature of the prodrug and the slow rate of hydrolysis of the chemical linkage as described above, should high levels of drug be administered either accidentally or intentionally, the prodrug will be cleared by metabolic and/or excretory pathways prior to releasing large amounts of the stimulant and without being absorbed and reaching the bloodstream as the intact prodrug. Also, slow, attenuated release of amphetamine (or another stimulant) over an extended period should alleviate or diminish drug induced side-effects that can limit or terminate amphetamine therapy. These side effects include increase in the heart and respiration rates, increased blood pressure, dilation of the pupils of the eyes, and decreased appetite (Adderall® Label, Dexedrine® Label).

Other side effects include anxiety, blurred vision, sleeplessness, and dizziness. Also, amphetamines are powerful psychostimulants and are prone to substance abuse ([a] Maxwell, J. C.; et al. The prevalence of methamphetamine and amphetamine abuse in North America: a review of the indicators, 1992-2007. Drug and Alcohol Review 2008, 27, 229-235. [b] Amphetamine-type stimulants: a report from the WHO meeting on amphetamines, MDMA and other psychostimulants, Geneva, 12-15 Nov. 1996).

Substance abuse of stimulants is often characterized by an escalation of events. First, a substantial “rush” or high may be obtained from increasing oral dosages. Due to the properties of homoarginine amphetamine prodrugs, these potential routes for abuse can be mitigated via the polar nature of the prodrug. That is, once administered at higher than therapeutic levels, the body will not absorb and subsequently excrete any remaining prodrug without breakdown into amphetamine. After oral amounts exceed an attainable amount, other routes can be explored including smoking, snorting, or injection. In accordance with the presently described technology, release of amphetamine or another stimulant would only occur under desired physiological conditions. Preferably, other routes of administration (e.g., intranasal or intravenous) do not break the prodrug down to any appreciable extent. Also preferably, external means (chemical, enzymatic or other) will not break the prodrug down to any appreciable extent either. The breakdown ratio of the prodrug that can be achieved through external means is preferably less than about 50%, alternatively less than about 25%, alternatively less than about 20%, alternatively less than about 10%.

The presently described technology utilizes covalent modification of amphetamine by homoarginine to decrease its potential for causing behavioral deterioration or the rebound effect. It is believed that since the amphetamine is covalently modified to form the homoarginine conjugate of the present technology and releases slowly over the entire length of the day, little or no rebound effect can occur due to the slow continuous release of the active ingredient/drug/therapeutic component.

Compounds, compositions and methods of the presently described technology are also believed to provide reduced potential for rebound, reduced potential for abuse or addiction, and/or improve amphetamine's stimulant related toxicities. By limiting the blood level spike, doses are kept at levels required for a clinically significant effect without the unnecessary levels administered with other therapies. It is widely held that these spikes in blood levels can lead to cardiovascular toxicity in the form of higher blood pressure and rapid heart rate in addition to the euphoria encountered in drug abuse. Also, with a full day therapy, the risk of re-dosing is lowered, thus preventing additional toxicities or drug abuse issues. For example, extended release formulations of methylphenidate have shown to reduce abuse liability compared to instant release formulations (Parasrampuria, D. A.; et al. Assessment of Pharmacokinetics and Pharmacodynamic Effects Related to Abuse Potential of a Unique Oral Osmotic-Controlled Extended-Release Methylphenidate Formulation in Humans. The Journal of Clinical Pharmacology 2007, 47(12), 1476-1488).

The homoarginine amphetamine prodrugs of the presently described technology could be used for any condition requiring the stimulation of the central nervous system (CNS). These conditions include, for example, attention deficit hyperactivity disorder (ADHD), attention deficit disorder (ADD), obesity, narcolepsy, appetite suppressant, depression, anxiety, withdrawals (e.g., alcohol withdrawals or drug withdrawals), and wakefulness. Some stimulants such as amphetamine have also demonstrated usefulness in treating stimulant (e.g., cocaine, methamphetamine) abuse and addiction. Amphetamine stimulants have also been used extensively to improve battle field alertness and to combat fatigue.

One or more embodiments of the present technology provide amphetamine compositions which allow amphetamine to be therapeutically effective when delivered at the proper dosage but reduce the rate of absorption or extent of bioavailability of amphetamine when given at doses exceeding those within the therapeutic range of amphetamine.

In one or more embodiments, the amphetamine prodrug compositions of the present technology have substantially lower toxicity compared to unconjugated amphetamine or the amphetamine conjugated with a standard amino acid. In one or more embodiments, the amphetamine prodrug compositions of the present technology can reduce or eliminate the possibility of overdose by oral administration. For example, the enzymes responsible for the hydrolysis of homoarginine-amphetamine could become saturated by a large amount of the prodrug and would consequently not be able to efficiently hydrolyze the conjugate. This could limit the exposure to free amphetamine while the pharmacologically inactive prodrug would be eliminated and excreted without serious adverse effects.

In one or more embodiments, the homoarginine amphetamine prodrugs of the present technology may further comprise a polymer blend which comprises a hydrophilic polymer and/or a water-insoluble polymer. The polymers may be used according to industry standards to further enhance the sustained release/abuse resistant properties of the amphetamine prodrug of the present technology without reducing the abuse resistance. For instance, a composition might include: about 70% to about 100% amphetamine prodrug of the present technology by weight, from about 0.01% to about 10% of a hydrophilic polymer (e.g. hydroxypropyl methylcellulose), from about 0.01% to about 2.5% of a water-insoluble polymer (e.g. acrylic resin), from about 0.01% to about 1.5% of additives (e.g. magnesium stearate), and from about 0.01% to about 1% colorant by weight.

Hydrophilic polymers suitable for use in the sustained release formulations include one or more natural or partially or totally synthetic hydrophilic gums such as acacia, gum tragacanth, locust bean gum, guar gum, or karaya gum, modified cellulosic substances such as methylcellulose, hydroxymethylcellulose, hydroxypropyl methylcellulose, hydroxypropyl cellulose, hydroxyethylcellulose, carboxymethylcellulose; proteinaceous substances such as agar, pectin, carrageen, and alginates; and other hydrophilic polymers such as carboxypolymethylene, gelatin, casein, zein, bentonite, magnesium aluminum silicate, polysaccharides, modified starch derivatives, and other hydrophilic polymers known to those of skill in the art, or a combination of such polymers. These hydrophilic polymers gel and would dissolve slowly in aqueous acidic media thereby allowing the amphetamine prodrug to diffuse from the gel in the stomach. When the gel reaches the intestines it would dissolve in controlled quantities in the higher pH medium to allow further sustained release. Preferred hydrophilic polymers are the hydroxypropyl methylcelluloses such as those manufactured by The Dow Chemical Company and known as Methocel ethers, such as Methocel E1OM.

Other formulations according to one or more embodiments of the present technology may further comprise pharmaceutical additives including, but not limited to, lubricants such as magnesium stearate, calcium stearate, zinc stearate, powdered stearic acid, hydrogenated vegetable oils, talc, polyethylene glycol, and mineral oil; colorants such as Emerald Green Lake, FD&C Red No. 40, FD&C Yellow No. 6, D&C Yellow No. 10, or FD&C Blue No. 1 and other various certified color additives (See 21 CFR, Part 74); binders such as sucrose, lactose, gelatin, starch paste, acacia, tragacanth, povidone, polyethylene glycol, Pullulan and corn syrup; glidants such as colloidal silicon dioxide and talc; surface active agents such as sodium lauryl sulfate, dioctyl sodium sulfosuccinate, triethanolamine, polyoxyethylene sorbitan, poloxalkol, and quaternary ammonium salts; preservatives and stabilizers; excipients such as lactose, mannitol, glucose, fructose, xylose, galactose, sucrose, maltose, xylitol, sorbitol, chloride, sulfate and phosphate salts of potassium, sodium, and magnesium; and/or any other pharmaceutical additives known to those of skill in the art. In one preferred embodiment, a sustained release formulation of the present technology further comprises magnesium stearate and Emerald Green Lake.

The compositions of the present technology, which comprises at least one homoarginine amphetamine prodrug of the present technology, can be further formulated with excipients, and may be manufactured according to any appropriate method known to those of skill in the art of pharmaceutical manufacture. For instance, the prodrug and a hydrophilic polymer may be mixed in a mixer with an aliquot of water to form a wet granulation. The granulation may be dried to obtain hydrophilic polymer encapsulated granules of the stimulant prodrug. The resulting granulation may be milled, screened, then blended with various pharmaceutical additives such as, for example, water insoluble polymers, and/or additional hydrophilic polymers. The formulation may then be tableted and may further be film coated with a protective coating which rapidly dissolves or disperses in gastric juices.

It should be noted that the above additives are not required for the homoarginine amphetamine prodrug composition of the present technology to have sustained release and abuse resistance properties. The homoarginine amphetamine prodrug of the present technology itself can control the release of the stimulant into the digestive tract over an extended period of time resulting in an improved profile when compared to immediate release combinations and prevention of abuse without the addition of the above additives. In one or more embodiments of the present technology, no further sustained release additives are required to achieve a blunted or reduced pharmacokinetic curve (e.g., reduced euphoric effect) while achieving therapeutically effective amounts of amphetamine release when taken orally.

The compounds and compositions of the presently described technology can be formulated into and administered by a variety of dosage forms, preferably, through any oral routes of delivery. Once administered, the prodrugs will release amphetamine under digestive conditions. Any biologically-acceptable dosage form known to persons of ordinary skill in the art, now or in the future, and combinations thereof, are contemplated for use with the present technology. Examples of preferred dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, troches, lozenges, chewable lozenges, beads, powders, granules, particles, microparticles, dispersible granules, cachets, thin strips, oral films, transdermal patches, and combinations thereof. Preferred dosage forms include, but are not limited to, capsules, thin strips, and solution formulations.

Formulations of the present technology suitable for oral administration can be presented as discrete units, such as capsules, caplets, oral thin films or strips, or tablets. These oral formulations also can comprise a solution or a suspension in an aqueous liquid or a non-aqueous liquid. The formulation can be an emulsion, such as an oil-in-water liquid emulsion or a water-in-oil liquid emulsion. The oils can be administered by adding the purified and sterilized liquids to a prepared enteral formula, which can then be placed in the feeding tube of a patient who is unable to swallow.

If the capsule form is chosen, for example, excipients used in the capsule formulation could be broken up into four separate groups: bulk agent/binder, disintegrant, lubricant and carrier. A preferred capsule formulation comprises from about 50% to about 90% by weight of a bulk agent such as various types of microcrystalline cellulose, from about 1% to about 5% by weight of a disintegrant such as croscarmellose sodium, from about 0.5% to about 2.5% of a lubricant such as magnesium stearate or other fatty acid salts. The carrier can be either hard gelatin capsules, and preferably use the smaller sized ones such as #3 or #4 hard gelatin capsules.

Soft gel or soft gelatin capsules may be prepared, for example, by dispersing the formulation of the present technology in an appropriate vehicle (vegetable oils are commonly used) to form a high viscosity mixture. This mixture can then be encapsulated with a gelatin based film using technology and machinery known to those in the soft gel industry. The individual units so formed are then dried to constant weight.

Chewable tablets, for example, may be prepared by mixing the formulations of the present technology with excipients designed to form a relatively soft, flavored, tablet dosage form that is intended to be chewed rather than swallowed. Conventional tablet machinery and procedures, that is both direct compression and granulation, i.e., or slugging, before compression, can be utilized. Those individuals involved in pharmaceutical solid dosage form production are versed in the processes and the machinery used as the chewable dosage form is a very common dosage form in the pharmaceutical industry.

Film-coated tablets, for example, may be prepared by coating tablets using techniques such as rotating pan coating methods or air suspension methods to deposit a contiguous film layer on a tablet.

Compressed tablets, for example, may be prepared by mixing the formulation of the present technology with excipients intended to add binding qualities to disintegration qualities. The mixture can be either directly compressed or granulated then compressed using methods and machinery known to those in the industry. The resultant compressed tablet dosage units are then packaged according to market need, i.e., unit dose, rolls, bulk bottles, blister packs, etc.

One preferred formulation of the homoarginine amphetamine prodrugs is a fast dissolving oral film or thin strip. The water solubility for homoarginine amphetamine is extremely high, making it particularly suited for an oral thin strip dosage form. For example, the water solubility of l-homoarginine-d-amphetamine dihydrochloride is greater than 5,000 mg/ml. Such high water solubility allows homoarginine amphetamine prodrugs to be formulated into fast dissolving oral thin films or strips without the need for complex formulations or ingredients to enhance solubility. In addition, as the prodrug releases amphetamine gradually over time, additional excipients and formulations are not necessary for once daily dosing and would not need to be added to the film. Methods and other ingredients needed to make oral films or thin strips are known in the art. Potential film forming agents include pullulan, hydroxypropylmethyl cellulose, hydroxypropyl cellulose, polyvinyl pyrrolidone, polyvinyl alcohol, sodium alginate, polyethylene glycol, xanthan gum, tragacanth gum, guar gum, acacia gum, Arabic gum, polyacrylic acid, amylase, starch, dextrin, pectin, chitin, chitosin, levan, elsinan, collagen, gelatin, zein, gluten, soy protein isolate, whey protein isolate, casein, and mixtures thereof.

Also, saliva stimulating agents, plasticizing agents, cooling agents, surfactants, emulsifying agents, thickening agents, binding agents, sweeteners, flavoring, coloring agents, preservatives, or taste masking resins may be employed in the oral films or thin strips. Preferred agents include: pullulan, triethanol amine stearate, methyl cellulose, starch, triacetin, polysorbate 80, xanthan gum, maltitol, sorbitol and glycerol.

The presently described technology also contemplates the use of biologically-acceptable carriers which may be prepared from a wide range of materials. Without being limited thereto, such materials include diluents, binders and adhesives, lubricants, plasticizers, disintegrants, colorants, bulking substances, flavorings, sweeteners and miscellaneous materials such as buffers and adsorbents in order to prepare a particular medicated composition.

Binders may be selected from a wide range of materials such as hydroxypropylmethylcellulose, ethylcellulose, or other suitable cellulose derivatives, povidone, acrylic and methacrylic acid co-polymers, pharmaceutical glaze, gums, milk derivatives, such as whey, starches, and derivatives, as well as other conventional binders known to persons skilled in the art. Exemplary non-limiting solvents are water, ethanol, isopropyl alcohol, methylene chloride or mixtures and combinations thereof. Exemplary non-limiting bulking substances include sugar, lactose, gelatin, starch, and silicon dioxide.

Preferred plasticizers may be selected from the group consisting of diethyl phthalate, diethyl sebacate, triethyl citrate, cronotic acid, propylene glycol, butyl phthalate, dibutyl sebacate, castor oil and mixtures thereof, without limitation. As is evident, the plasticizers may be hydrophobic as well as hydrophilic in nature. Water-insoluble hydrophobic substances, such as diethyl phthalate, diethyl sebacate and castor oil are used to delay the release of water-soluble vitamins, such as vitamin B6 and vitamin C. In contrast, hydrophilic plasticizers are used when water-insoluble vitamins are employed which aid in dissolving the encapsulated film, making channels in the surface, which aid in nutritional composition release.

It should be understood that in addition to the ingredients particularly mentioned above, the formulations of the present technology can include other suitable agents such as flavoring agents, preservatives and antioxidants. Such antioxidants would be food acceptable and could include, for example, vitamin E, carotene, BHT or other antioxidants known to those of skill in the art.

Other compounds which may be included are, for example, medically inert ingredients, e.g., solid and liquid diluent, such as lactose, dextrose, saccharose, cellulose, starch or calcium phosphate for tablets or capsules, olive oil or ethyl oleate for soft capsules and water or vegetable oil for suspensions or emulsions; lubricating agents such as, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

For oral administration, fine powders or granules containing diluting, dispersing and/or surface-active agents may be presented in a draught, in water or a syrup, in capsules or sachets in the dry state, in a non-aqueous suspension wherein suspending agents may be included, or in a suspension in water or a syrup. Where desirable or necessary, flavoring, preserving, suspending, thickening or emulsifying agents can be included.

Liquid dispersions for oral administration may be syrups, emulsions or suspensions. The syrups may contain as a carrier, for example, saccharose or saccharose with glycerol and/or mannitol and/or sorbitol. The suspensions and the emulsions may contain a carrier, for example a natural gum, agar, sodium alginate, pectin, methylcellulose, carboxymethylcellulose or polyvinyl alcohol.

The dose range for adult or pediatric human beings will depend on a number of factors including the age, weight and condition of the patient. Suitable oral dosages of the prodrugs of one stimulant of the presently described technology can be the equivalents of those typically found in treatments using that stimulant. For example, typical dosages for amphetamine salts can range from about 1 mg to about 100 mg, although higher dosages may be approved at later dates. Preferred doses of the prodrug are doses equimolar to amphetamine freebase in the range from about 5 mg to about 40 mg. Even more preferred doses of the prodrug are doses equimolar to amphetamine freebase in the range from about 9 mg to about 30 mg. For example, doses of a preferred homoarginine amphetamine dichloride prodrug in the range of about 25 mg to about 75 mg would provide an amphetamine freebase content in the preferred range of about 9 mg to about 30 mg. Using the molecular weight of the prodrug of the present technology, the release percentage (% release) of amphetamine from the prodrug and desired dosage forms of the required amphetamine, the following equation can be generated:

grams of a prodrug needed=(dosage/molecular weight of amphetamine)(% release)(molecular weight of the prodrug)

Tablets, capsules, and other forms of presentation provided in discrete units conveniently contain a daily dose, or an appropriate fraction thereof, of one or more of the prodrug compounds of the invention. For example, the units may contain from about 1 mg to about 1000 mg, alternatively from about 5 mg to about 500 mg, alternatively from about 5 mg to about 250 mg, alternatively from about 10 mg to about 100 mg of one or more of the prodrug compounds of the presently described technology. Preferred units of the prodrug are dose units equimolar to amphetamine freebase in the range from about 9 mg to about 27 mg.

It is also possible for the dosage form of the present technology to combine any forms of release known to persons of ordinary skill in the art. These conventional release forms include immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics and combinations thereof is known in the art.

Compositions of the present technology may be administered in a partial, i.e., fractional dose, one or more times during a 24 hour period, a single dose during a 24 hour period of time, a double dose during a 24 hour period of time, or more than a double dose during a 24 hour period of time. Fractional, double or other multiple doses may be taken simultaneously or at different times during the 24 hour period. The doses may be uneven doses with regard to one another or with regard to the individual components at different administration times.

Likewise, the compositions of the present technology may be provided in a blister pack, individual foil packages of a child-proof nature or other such pharmaceutical package. Further, the compositions of the present technology may further include or be accompanied by indicia allowing individuals to identify the compositions as products for a prescribed treatment. The indicia may additionally include an indication of the above specified time periods for administering the compositions. For example, the indicia may be time indicia indicating a specific or general time of day for administration of the composition, or the indicia may be a day indicia indicating a day of the week for administration of the composition. The blister pack, individual foil packages of a child-proof nature or other combination package may also include a second pharmaceutical product.

It will be appreciated that the pharmacological activity of the compositions of the present technology can be demonstrated using standard pharmacological models that are known in the art. Furthermore, it will be appreciated that the compositions of the present technology can be incorporated or encapsulated in a suitable polymer matrix or membrane for site-specific delivery, or can be functionalized with specific targeting agents capable of effecting site specific delivery. These techniques, as well as other drug delivery techniques, are well known in the art.

In one or more embodiments of the present technology, the solubility and dissolution rate of the composition can be substantially changed under different physiological conditions encountered, for example, in the intestine, at mucosal surfaces, or in the bloodstream. In one or more embodiments of the present technology, the solubility and dissolution rate of the composition can substantially decrease the bioavailability of the amphetamine, particularly at doses above those intended for therapy. In one embodiment of the present technology, the decrease in bioavailability occurs upon intranasal administration. In another embodiment, the decrease in bioavailability occurs upon intravenous or intra-arterial administration. In another embodiment, the decrease in bioavailability occurs upon subcutaneous administration. In another embodiment, the decrease in bioavailability occurs upon intramuscular administration.

In another embodiment, the decrease in bioavailability occurs upon rectal administration. In another embodiment, the decrease in bioavailability occurs upon intravaginal administration. In another embodiment, the decrease in bioavailability occurs upon intracavernous injection. In another embodiment, the decrease in bioavailability occurs upon intraperitoneal injection. In another embodiment, the decrease in bioavailability occurs upon inhalation. In another embodiment, the decrease in bioavailability occurs upon transdermal administration. In another embodiment, the decrease in bioavailability occurs upon buccal or sublingual administration.

For each of the described embodiments of the present technology, one or more of the following characteristics can be realized: The cardiovascular toxicity of the homoarginine-amphetamine prodrug is substantially lower than that of the unconjugated amphetamine. The covalently bound homoarginine reduces or eliminates the possibility of behavioral deterioration or the rebound effect. The covalently bound homoarginine amphetamine reduces or eliminates the possibility of abuse by intranasal administration. The covalently bound homoarginine amphetamine reduces the possibility of abuse by various forms of injection. The covalently bound homoarginine amphetamine reduces the possibility of abuse by inhalation. The covalently bound homoarginine amphetamine reduces the possibility of abuse by transdermal administration. The covalently bound homoarginine amphetamine reduces the possibility of abuse by buccal or sublingual administration. The covalently bound homoarginine amphetamine reduces the possibility of abuse by intravaginal or rectal administration.

Another embodiment provides a method for safely delivering amphetamine or another stimulant composition providing a therapeutically effective amount of at least one homoarginine prodrug of stimulant of the present technology wherein the homoarginine can reduce the rate of absorption of amphetamine or another stimulant as compared to delivering the unconjugated stimulant, for example.

Another embodiment provides a method for reducing stimulant toxicity by providing a patient with at least one homoarginine prodrug of the stimulant of the present technology, wherein the homoarginine moiety can increase the rate of clearance of the pharmacologically active stimulant (i.e., released stimulant such as amphetamine) when given at doses exceeding those within the therapeutic range of the stimulant.

Another embodiment provides a method for reducing stimulant toxicity by providing a patient with at least one homoarginine stimulant prodrug of the present technology, wherein the homoarginine moiety can provide a serum release curve which does not increase above the stimulant's toxicity level when given at doses exceeding those within the therapeutic range for the unconjugated stimulant.

Another embodiment provides a method for reducing bioavailability of a stimulant composition providing at least one homoarginine stimulant prodrug of the present technology, wherein the stimulant prodrug can maintain a steady-state serum release curve which provides a therapeutically effective bioavailability but prevents spiking or increased blood serum concentrations compared to unconjugated stimulant when given at doses exceeding those within the therapeutic range for the unconjugated stimulant, for example.

Another embodiment provides a method for preventing a Cₘₐₓ or equivalent Cₘₐₓ spike for amphetamine or another stimulant while still providing a therapeutically effective bioavailability curve comprising the step of administering to a patient at least one homoarginine, prodrug of amphetamine or another stimulant of the present technology.

Another embodiment provides a method for preventing a toxic release profile in a patient by administering to a patient at least one homoarginine stimulant prodrug of the present technology, wherein the stimulant prodrug can maintain a steady-state serum release curve which provides a therapeutically effective bioavailability but prevents spiking or increased blood serum concentrations compared to unconjugated stimulant, particularly when taken at doses above prescribed amounts.

Another embodiment of the present technology is a method for reducing or preventing abuse of a stimulant by providing, administering, or prescribing a composition to a patient in need thereof, wherein said composition comprises at least one homoarginine stimulant prodrug of the present technology such that the pharmacological activity of the stimulant is decreased when the composition is used in a manner inconsistent with the manufacturer's instructions.

Another embodiment of the present technology is a method for reducing or preventing abuse of a stimulant such as amphetamine by providing at least one homoarginine prodrug of the stimulant of the present technology, wherein said prodrug comprises the stimulant covalently attached to homoarginine such that the pharmacological activity of the stimulant is substantially decreased when the composition is used in a manner inconsistent with the manufacturer's instructions.

Another embodiment of the present technology is a method of preventing behavioral deterioration or the rebound effect of amphetamine or stimulant treatment by providing, administering, or prescribing an amphetamine composition of the presently described technology to a patient in need thereof, wherein said composition comprises at least one homoarginine prodrug of amphetamine or a derivative thereof that can decrease the potential of behavioral deterioration or the rebound effect from amphetamine or stimulant treatment.

Another embodiment of the present technology is a method for reducing or preventing the euphoric effect of a stimulant by providing, administering, or prescribing to a human or animal in need thereof, a composition comprising at least one homoarginine stimulant prodrug of the present technology that can decrease the pharmacological activity of the stimulant when the composition is used in a manner inconsistent with the manufacturer's instructions.

Another embodiment of the present technology is a method for reducing or preventing the euphoric effect of a stimulant, comprising consuming a composition comprising at least one homoarginine stimulant prodrug of the present technology that can decrease the pharmacological activity of the stimulant when the composition is used in a manner inconsistent with the manufacturer's instructions. Preferably, the prodrug would not hydrolyze efficiently when administered via intranasal and intravenous routes, such that a dose equivalent to a therapeutic oral dose would not induce euphoria when snorted or injected.

Another embodiment of the present technology is any of the preceding methods wherein the stimulant composition used is adapted for oral administration, and wherein the stimulant prodrug is resistant to release the stimulant from the homoarginine moiety when the composition is administered parenterally, such as intranasally or intravenously. Preferably, the stimulant may be released from the homoarginine moiety in the presence of water and/or enzymes present in the stomach, intestinal tract, or blood serum. Optionally, the stimulant composition used may be in the form of a tablet, chewable tablet, orally dissolving tablet, capsule, oral solution, oral suspension, thin strip or other oral dosage form discussed herein. An oral thin strip dosage form is preferred, however, because such a dosage form is easily administered and is likely to increase patient compliance, especially in children.

For one or more of the recited methods, the composition of the present technology used may yield a therapeutic effect without substantial euphoria. Preferably, the stimulant composition of the present technology can provide a therapeutically equivalent AUC when compared to the stimulant alone but does not provide a Cₘₐₓ which results in euphoria or an equivalent Cₘₐₓ.

Another embodiment of the present technology is a method for reducing or preventing abuse of stimulants such as amphetamine or derivatives thereof comprising orally administering a stimulant prodrug composition of the present technology to a patient, wherein said composition comprises at least one homoarginine stimulant prodrug of the present technology that can decrease the pharmacological activity of the stimulant when the composition is used in a manner inconsistent with the manufacturer's instructions.

Another embodiment is a method for reducing or preventing the euphoric effect of a stimulant comprising orally administering a stimulant prodrug composition of the present technology to a patient in need thereof, wherein said composition comprises at least one homoarginine prodrug of the stimulant of the present technology that can decrease the pharmacological activity of the stimulant when the composition is used in a manner inconsistent with the manufacturer's instructions.

In one embodiment, the cardiovascular toxicity or stress of the homoarginine amphetamine conjugate may be lower than that of the amphetamine when the amphetamine is delivered in its unconjugated state.

The presently described technology and its advantages will be better understood by reference to the following examples. These examples are provided to describe specific embodiments of the present technology. By providing these specific examples, the applicants do not limit the scope and spirit of the present technology. It will be understood by those skilled in the art that the full scope of the presently described technology encompasses the subject matter defined by the claims appending this specification, and any alterations, modifications, or equivalents of those claims.

### Example 1

### Comparative Animal Study of Pharmacokinetic Parameters of Released d-Amphetamine Following Administration of a Polar Hydrophilic Prodrug of the Non-Standard Amino Acid Type (hArg-Amp) and a Standard Amino Acid Conjugate (Vyvanse™, Lys-Amp)

The pharmacokinetic parameters of d-amphetamine following oral administration of a non-standard amino acid conjugate of the present technology, homoarginine amphetamine, and a standard amino acid conjugate, Vyvanse™ (Lys-Amp), commercially available from Shire, Incorporated of Wayne, Pa. are studied in rats in this example. The homoarginine amphetamine conjugate used in this example is the hydrochloride salt of hArg-Amp. The results are recorded in the table below:

TABLE 1

| | Non-standard amino | Vyvanse ™ |
|---|---|---|
| Parameter | Acid % amp¹ | % total Amp² |
| AUC_{0-8 h} | 94% | 100% |
| AUC_{0-4 h} | 77% | 100% |
| AUC_{inf} | 95% | 100% |
| Cₘₐₓ | 76% | 100% |
| Tₘₐₓ | 400% | 100% |

¹Percent amphetamine released relative to Vyvanse ™ (at an equimolar concentration of amphetamine contained in the non-standard amino acid prodrug, homoarginine amphetamine, compared to the total amphetamine contained in Vyvanse ™)
²Percent amphetamine relative to 50 mg Vyvanse ™ dose

The study shows that the Cₘₐₓ of a prodrug of the preset technology is significantly lower than that of Vyvanse™, a standard amino acid conjugate of d-amphetamine, which can lead to lower cardiovascular effects (blood pressure, heart rate). Quick release (higher Cₘₐₓ) of amphetamine has already demonstrated significant increases in blood pressure and heart rate. In certain patient populations, these cardiovascular side effects can be dose limiting or can cause the termination of stimulant therapy.

The pharmacokinetic parameters of d-amphetamine following parental administration in rats of hArg-Amp and d-amphetamine are also studied. The study shows that little release of amphetamine (<25%) happens when hArg-Amp is taken through parental routes (intranasal, intravenous) potentially due to differences in enzymes encountered in the gut versus other routes. When Adderall XR® or other controlled release formulations of amphetamine are injected or snorted, the pharmacokinetic parameters of the amphetamine are significantly altered and an individual can use these changes to produce euphoria.

### Example 2

### Preparation of Boc-hArg(NO₂)-Amp

Boc-hArg(NO₂)—OH (2.667 g, 8 mmol) was dissolved in DMF (25 ml). EDCI (2.30 g, 12 mmol), NHS (1.012 g, 8.8 mmol), d-amphetamine (1.269 g, 9.6 mmol) and DIEA (1.138 g, 8.8 mmol) were then added sequentially. The clear reaction mixture was stirred at room temperature for 16 hrs. The reaction mixture was quenched with pH 3 water (150 ml), and the product was extracted with EtOAc (3×50 ml). The combined extracts were washed with pH 3 water followed by saturated NaCl. The EtOAc layer was dried over anhydrous MgSO₄. The product was recrystallized from EtOAc-Hexane two times to give 2.36 g of desired protected product.

The product was analyzed using ¹H NMR (DMSO-d₆) δ. The result shows 0.9-1.1 (m, 3H, Amp CH₃), 1.1-1.2 (m, 2H, hArg γ CH₂), 1.2-1.5 (m, 13H, Boc CH₃, hArg β, δ CH₂), 2.55-2.75 (m, 2H, Amp β CH₂), 3.1 (m, 2H, hArg ε CH₂), 3.75 (m, 1H, Amp α CH), 3.95 (m, 1H, hArg α CH), 6.65 (t, 1H, hArg guanidino NH), 7.1-7.3 (m, 5H, Amp Ar—H), 7.6-8.2 (br m, 2H, hArg guanidine NH and amide NH), 8.5 (br s, 1H, hArg NH—NO₂). These results are consistent with the proposed structure.

### Example 3

### Preparation of hArg-Amp.2HCl (l-homoarginine-d-amphetamine dihydrochloride)

Boc-hArg(NO₂)-Amp (1.5 g) was dissolved in HPLC grade MeOH (120 ml) and to the clear solution was added the Pd—C catalyst (10%, Aldrich). A small stir bar was placed in the flask and the reaction mixture was stirred under a slow stream of hydrogen overnight after incorporating the 5-6N HCl in 2-propanol solution (1.5 ml). After the overnight reaction, the solution was filtered and the solvent evaporated. The white crystalline product was dried under vacuum to give 1.61 g of the Boc-hArg-Amp intermediate product.

The product (1.6 g) was dissolved in 80 ml of HPLC grade MeOH, and 5-6N HCl in 2-propanol (3.2 mL) was added to the solution. The reaction mixture was stirred overnight, solvent removed and re-dissolved in minimum amount of MeOH. The final product was crashed out with MTBE, and dried under vacuum at 30° C. for about 20 hours to yield 1.12 g of a white powder.

The white powder was analyzed using ¹H NMR (DMSO-d₆) δ. The result shows 0.9-1.1 (m, 3H, Amp CH₃), 1.1-1.2 (m, 2H, hArg γ CH₂), 1.35 (m, 2H, hArg β CH₂), 1.55 (m, 2H, hArg δ CH₂), 2.75 (d, 2H, Amp β CH₂), 3.0 (m, 2H, hArg ε CH₂), 3.75 (m, 1H, Amp α CH), 4.05 (m, 1H, hArg α CH), 7.1-7.2 (m, 5H, Amp Ar—H), 7.2-7.8 (br m, 3H, amide NH, HCl), 8.0 (t, 1H, hArg guanidino NH), 8.2 (br s, 2H, amide or guanidino NH₂), 8.75 (d, 1H, amide NH); ¹³C NMR (DMSO-d₆) δ 21.08 (Amp CH₃), 21.36 (hArg γ), 28.23 (hArg δ), 32.28 (hArg β), 40.18 (Amp β), 42.19 (hArg ε), 46.88 (Amp α), 52.23 (hArg α), 126.54 (p-Ar), 128.52 (m-Ar), 129.60 (o-Ar), 139.34 (Ar), 157.61 (C═O), 167.95 (guanidino C); M+1=306. These results are consistent with the proposed structure.

B=0.1% TFA/MeCN; method: 0-15 min.: 85/15→60/40, 15-25 min.: 60/40→0/100; flow rate: 1 mL/min.; UV detection: 230 nm; retention time: 8.92.

### Example 4

### Alternative Preparation of Boc-hArg(NO₂)-Amp

A 50-L glass lined reactor was charged with 0.96 kg (1.0 eq) of Boc-l-hArg(NO₂)—OH, 0.37 kg (1.1 eq) of N-hydroxysuccinimide (NHS), 0.85 kg (1.5 eq) of N-(3-dimethylaminopropyl)-N′-ethylcarbodiimide hydrochloride (EDCI) and 5.6 kg of DMSO at 20° C. Slowly, 0.48 kg (1.2 eq) of d-amphetamine was charged into the solution while maintaining the temperature below 30° C. The mixture was cooled to 24.5° C. and 0.33 kg (1.1 eq) of 4-methylmorpholine was charged into the reactor. The solution was agitated while maintaining a reaction temperature of 15-30° C.

After a total of 3.8 hours of reaction time, 22.42 kg of 2-methyltetrahydrofuran (2-MeTHF) was charged into the solution and the mixture was cooled to 13.5° C. A 10% acetic acid solution (2.01 kg acetic acid in 17.16 kg of water) was slowly charged into the reactor over a 10 min. period. The temperature did not exceed 24.8° C. during the addition. The contents were agitated for 5 min. at 23° C. and then allowed to phase separate for 10 min. The aqueous layer (25.9 kg) was drained and a solution of 5% sodium bicarbonate (1.30 kg of sodium bicarbonate in 23.0 kg of water) was charged into the reactor over a 10 min. period with the temperature not exceeding 22.2° C. The mixture was agitated for 5 min. at 21.1° C. and then allowed to phase separate for 20 min. The aqueous layer (27.4 kg) was drained and 11.94 kg of water was charged to the remaining content. The mixture was agitated for 5 min. at 23° C. and then allowed to phase separate for 10 min. The aqueous layer (13.6 kg) was drained.

A Dean Stark apparatus was attached to the reactor and the temperature of the contents was adjusted to 76.5° C. to remove water from the mixture by azeotropic distillation. Approximately 11 L (including 1075 mL of water) of solvents were removed from the original 21 L of solution volume over a period of 17 hours. The remaining mixture was cooled to 26.0° C. over a period of 1.1 hour while keeping the precipitate suspended through agitation. While agitating the temperature of the contents was subsequently adjusted to 5.0° C. and then held for 4.8 hours. The suspension was filtered through an 18 inch Büchner funnel. The filter cake was washed with 2.33 kg of 2-methyltetrahydrofuran and then transferred into a new dedicated filter reactor. The solids were dried in the filter reactor under vacuum for 29 hours at 65° C. with a nitrogen purge. The yield was 0.81 kg (62%) of Boc-l-hArg(NO₂)-d-amphetamine from 0.96 kg of Boc-l-hArg(NO₂)—OH. HPLC-UV analysis indicated a purity of 93.6%.

### Example 5

### Alternative preparation of hArg-Amp.2 HCl

A 30 L glass reactor was purged with nitrogen for 5 min. and then charged with 1.01 kg (1.0 eq) of Boc-l-hArg(NO₂)-Amp, 0.50 kg (0.10 eq) of 10% palladium on carbon (50% wet), 22.36 kg of methanol and 0.56 kg (2.5 eq) of 36% hydrochloric acid. The temperature of the contents was adjusted to 19.2° C. and agitation was started. A vacuum was pulled three times to remove air from the headspace until the mixture started to bubble and vacuum was broken each time with nitrogen. A vacuum was pulled three times and was broken each time with hydrogen. A balloon filled with hydrogen was attached to the reactor via a gas manifold and monitored and refilled as necessary to maintain appropriate hydrogen pressure throughout the reaction. The reaction was complete after 5.5 hours at which time no residual starting material was detected.

A filter pad was prepared in a new filter reactor containing 0.50 kg of filter aid Celatom Fw-60 and a top-layer of 0.039 kg of carbon. The reaction mixture was filtered through the filter reactor. The 30 L reactor was rinsed with 1.38 kg of methanol and filtered through the filter reactor. The combined filtrates were transferred into a 50 L glass reactor through a 45 micron filter. The container holding the original filtrate was rinsed with 1.22 kg of methanol and the wash was charged into the same 50 L reactor through the 45 micron filter.

The reactor containing the solution of Boc-l-hArg-d-amphetamine intermediate was purged with nitrogen for 5 min. The nitrogen sweep continued throughout the deprotection process (to facilitate removal of generated isobutylene). The reactor was charged with 1.35 kg (5.9 eq) of 36% hydrochloric acid. The temperature of the contents was adjusted to 65.6° C. for 3 hours. The temperature of the contents was adjusted to 28° C. The mixture was transferred from the reactor into a 20 L rotary evaporator (rotavap) and solvents were evaporated at 44.9° C. and 44 mbar for 16.9 hours. The remaining content was cooled to 26.4° C.

The rotavap bulb was charged with 3.70 kg of water to dissolve the residue. The resulting solution was transferred from the rotavap into a clean 50 L glass reactor and the temperature of the contents was adjusted to 21.9° C. The rotavap bulb was rinsed with 0.59 kg of water and the wash was added to the 50 L reactor. The reactor was charged with 7.32 kg of methyl tert-butyl ether (MTBE). The mixture was agitated for 8 min. at 21.4° C. and then allowed to phase separate for 10 min. The organic layer (MTBE) was discarded and the reactor charged again with 7.30 kg of MTBE. The contents were agitated for 5 min. at 24.3° C. and then allowed to phase separate for 20 min. The organic layer was discarded and the reactor charged a third time with 7.31 kg of MTBE. The mixture was agitated for 6 min. at 22.6° C. and then allowed to phase separate for 24 min. The organic layer was discarded and the remaining aqueous stream was transferred into a clean rotavap bulb.

To the rotavap bulb was then added 4.72 kg of ethanol (200 Proof) to accelerate the removal of water. The solution was concentrated for 3.3 hours at 55.0° C. and 47 mbar. The rotavap bulb was charged a second time with 4.70 kg of ethanol. Solvents were evaporated for 21.5 hours at 52.8° C. and 16 mbar. The rotavap bulb was charged a third time with 4.70 kg of ethanol. The solution was concentrated for 24.1 hours at 53.0° C. and 18 mbar. The rotavap bulb was charged a fourth time with 4.71 kg of ethanol. Solvents were evaporated for 19.2 hours at 53.1° C. and 21 mbar. The remaining solids were dried in the rotavap bulb for 96 hours at 70±5° C. The yield of l-hArg-d-amphetamine.2HCl was 0.83 kg (103%). Purity by HPLC-UV was 99.0%.

### Example 6

### Pharmacokinetic Study of hArg-Amp vs. Lys-Amp

Male Sprague-Dawley rats were fasted overnight and dosed by oral gavage with either l-homoarginine-d-amphetamine (hArg-Amp) or l-lysine-d-amphetamine (Vyvanse™, Lys-Amp). Water was provided ad libitum. Doses were calculated at an equivalent 1.5 mg/kg freebase equivalent of d-amphetamine. Plasma concentrations of d-amphetamine were measured using ELISA (Neogen Corp. Lexington, Ky.).

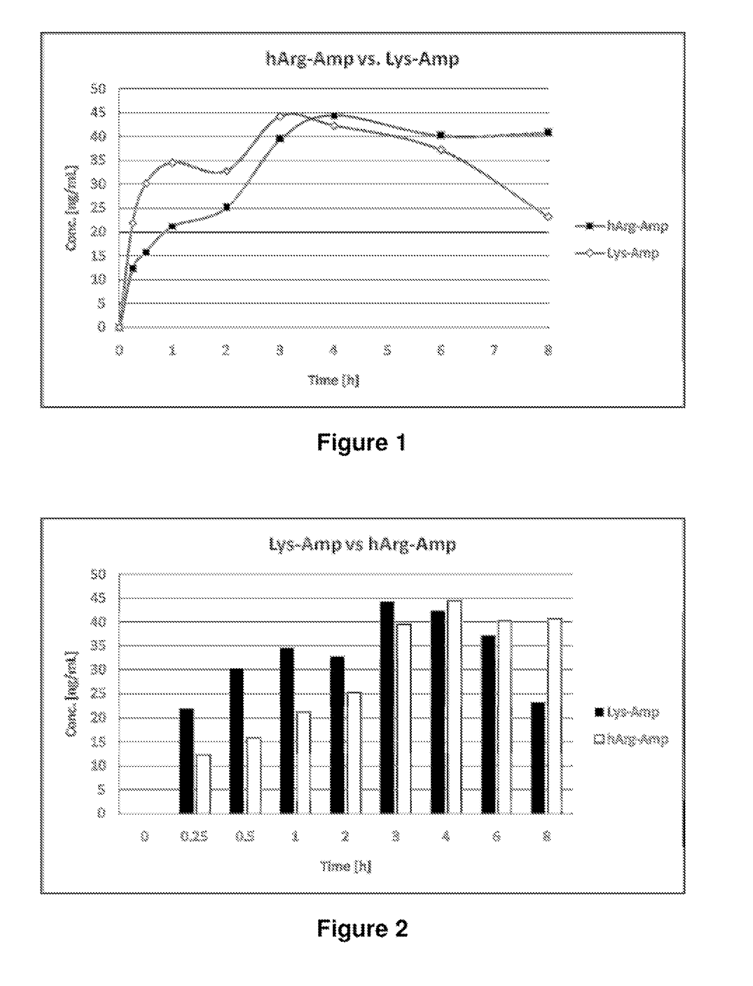

Mean plasma concentration curves (n=5) of d-amphetamine released by l-homoarginine-d-amphetamine or l-lysine-d-amphetamine are shown in FIG. 1. Pharmacokinetic(PK) parameters of this study are listed in Table 2.

TABLE 2
Pharmacokinetic Properties of hArg-Amp and Lys-Amp

| Vehicle | % AUC | Tmax | Cmax | % Tmax | % Cmax |
|---|---|---|---|---|---|
| Lys-Amp | 100% | 3 h | 44 ng/ml | 100% | 100% |
| hArg-Amp | 99% | 4 h | 44 ng/ml | 133% | 100% |

This pharmacokinetic (PK) study clearly demonstrates a shift in the Tₘₐₓ for the homoarginine amphetamine prodrug compared to the standard amino acid (Lys-Amp).

FIGS. 2-4 represent different ways to view the data reflected in FIG. 1 and Table 2. As further discussed below, these figures highlight the differences of hArg-Amp over Lys-Amp during the first several hours.

FIG. 2 demonstrates the relative blood levels of d-amphetamine released from both Lys-Amp and hArg-Amp. The graph shows that equivalent blood levels do not occur until later time points and that blood levels do not appear to spike or have a more significant Cₘₐₓ than Lys-Amp. The amount of d-amphetamine released from hArg-Amp is gradual and maintains a more steady concentration over the duration of the study than did Lys-Amp. In contrast, Lys-Amp blood levels of released d-amphetamine “spiked” at 3 hours and cleared more quickly than the blood levels obtained from hArg-Amp.

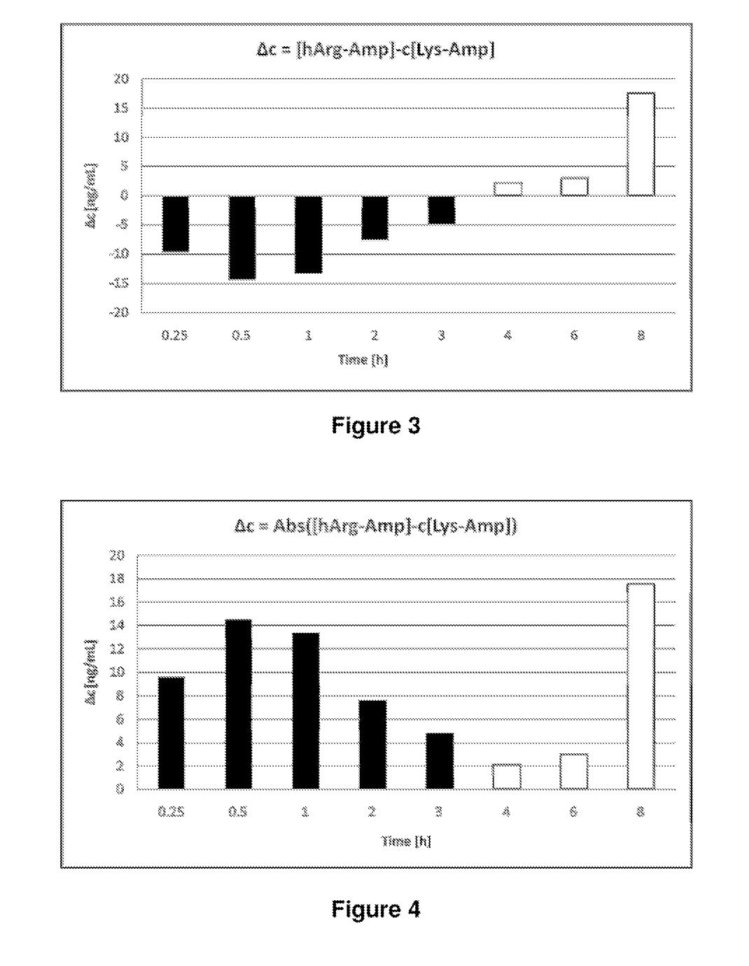

FIGS. 3 and 4 show the difference in blood levels obtained from the study described in FIG. 2. As is shown, the initial blood levels for both conjugates (Lys-Amp and hArg-Amp) are very different, with hArg-Amp releasing amphetamine at a more gradual rate. These differences in blood levels become less during the more critical duration of action for stimulant treatments and more importantly, the differences are greater again at later time points suggesting that hArg-Amp maintains a more consistent dose of amphetamine when compared to Lys-Amp. The longer duration of release for hArg-Amp would suggest a much lower opportunity for behavioral deterioration to occur.

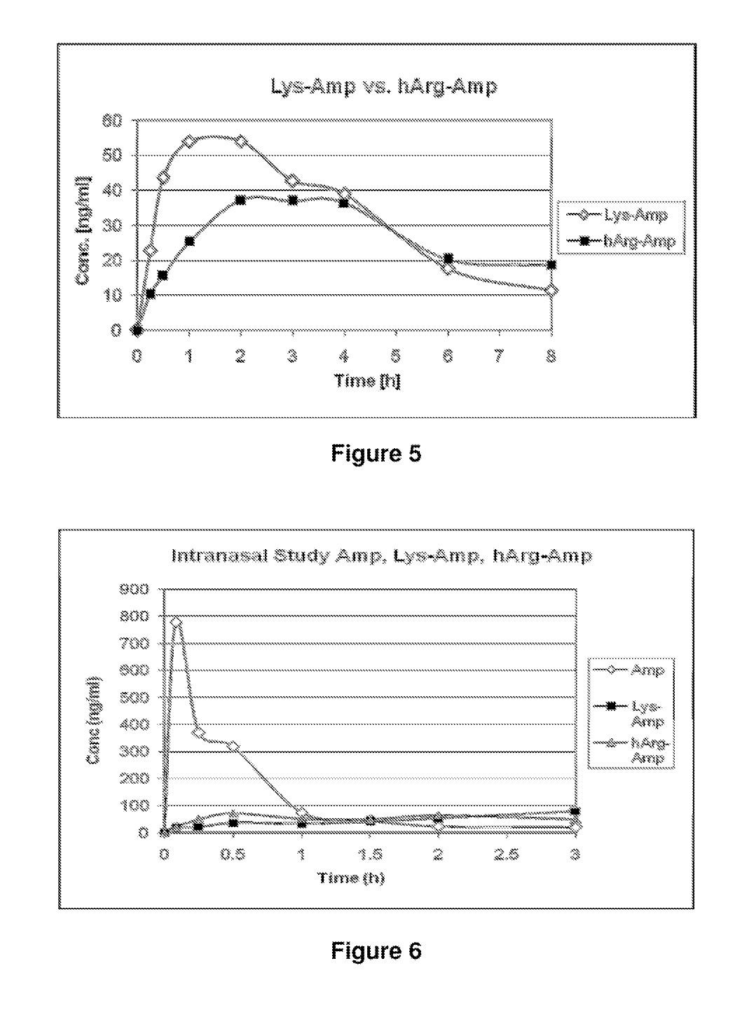

Other oral studies have been conducted in a similar fashion and are summarized in Table 3 below. The average PK results for four (4) oral studies (n=30 per vehicle) are recorded in FIG. 5:

TABLE 3
Average Results of 6 Oral Studies in Rats (n = 30 per compound)

| Vehicle | % AUC | Tmax | % Tmax | % Cmax | % AUC 0-4 h |
|---|---|---|---|---|---|
| Lys-Amp | 100% | 1 h | 100% | 100% | 100% |
| hArg-Amp | 81% | 2-4 h | 200-400% | 69% | 67% |

### Example 7

### Intranasal Study of Amp, Lys-Amp and hArg-Amp

Male Sprague-Dawley rats were fasted overnight and dosed by intranasal administration with either hArg-Amp, Lys-Amp or d-amphetamine. Doses were calculated at an equivalent 1.5 mg/kg freebase equivalent of d-amphetamine. Plasma concentrations of d-amphetamine were measured using ELISA. Mean plasma concentration curves (n=5) of d-amphetamine released by hArg-Amp or Lys-Amp are shown in FIG. 6. Pharmacokinetic parameters of this study are listed in Table 4. No significant release (<50%) was observed in either hArg-Amp or Lys-Amp and less release was observed within the first hour of administration (<25%). Observed levels from Lys-Amp are significantly higher than previously published data.

TABLE 4
Intranasal Properties of d-Amp, hArg-Amp and Lys-Amp

| Vehicle | % AUC | Tmax | Cmax | % Tmax | % Cmax |
|---|---|---|---|---|---|
| d-amp | 100% | 5 m | 779 ng/ml | 100% | 100% |
| hArg-Amp | 42% | 0.5 h | 71 ng/ml | 600% | 9% |
| Lys-Amp | 36% | 3 h | 79 ng/ml | 3600% | 10% |

### Example 8

### Intravenous Study of d-Amp, hArg-Amp, Lys-Amp

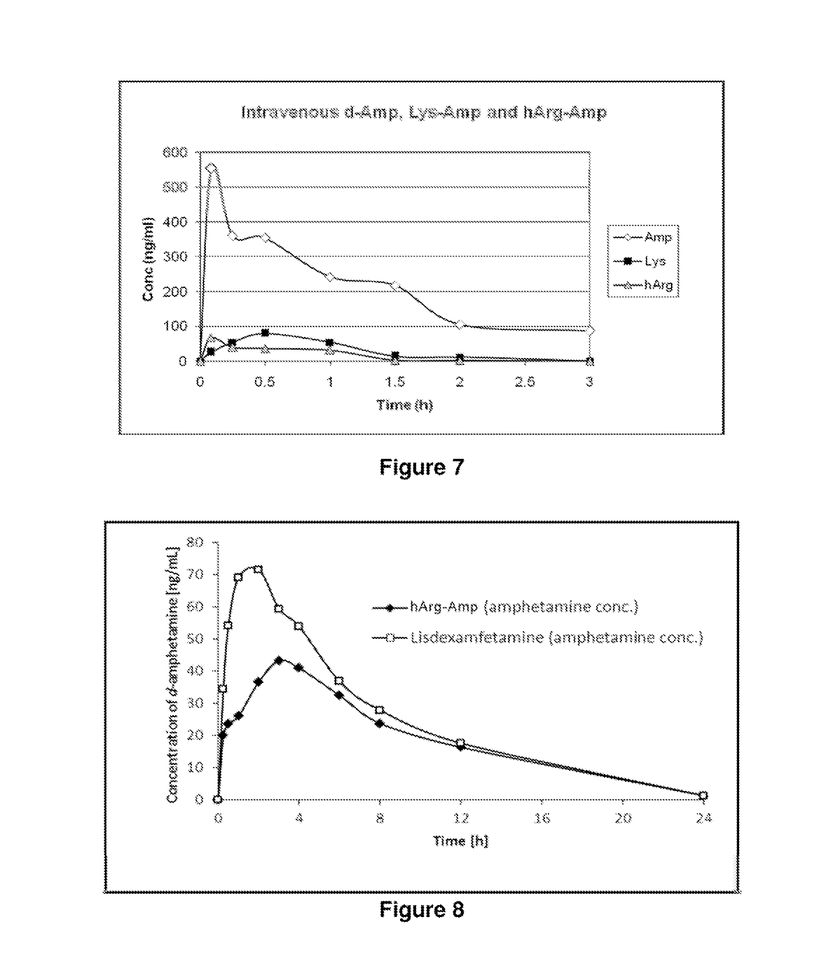

Male Sprague-Dawley rats were dosed by intravenous administration through the tail vein with hArg-Amp, Lys-Amp or d-amphetamine. Doses were calculated at an equivalent 1.5 mg/kg freebase equivalent of d-amphetamine. Plasma concentrations of d-amphetamine were measured using ELISA. Mean plasma concentration curves (n=5) of d-amphetamine released by hArg-Amp or Lys-Amp are shown in FIG. 7. Pharmacokinetic parameters of this study are listed in Table 5. No significant release (<15%) was observed in either hArg-Amp or Lys-Amp though hArg-Amp was significantly less. Observed levels from Lys-Amp are significantly higher than previously published data. The initial spike in d-amphetamine released from hArg-Amp cleared quickly.

TABLE 5
Intravenous Properties of d-Amp, hArg-Amp and Lys-Amp

| Vehicle | % AUC | Tmax | Cmax | % Tmax | % Cmax |
|---|---|---|---|---|---|
| d-amp | 100% | 5 m | 554 ng/ml | 100% | 100% |
| hArg-Amp | 8% | 5 m | 68 ng/ml | 100% | 12% |
| Lys-Amp | 14% | 15 m | 79 ng/ml | 100% | 14% |

Results of the studies in above examples clearly show an unexpected change in the oral pharmacokinetic properties by using homoarginine amphetamine conjugates. By using homoarginine as the group attached to amphetamine, the conjugates are able to shift Tₘₐₓ (earlier or later), modify curve shape, lower Cₘₐₓ, and raise Cₘₐₓ. In addition, the shift in Tₘₐₓ for hArg-Amp may be clinically significant in that many of the cardiovascular side effects and toxicity are related to Tₘₐₓ and Cₘₐₓ. The results demonstrate that by using homoarginine, a shift in the Tₘₐₓ, with a lower Cₘₐₓ occurs without changing AUC significantly. In addition, the slope of uptake of hArg-Amp vs. Lys-Amp appears to be more gradual thus leading to a slower onset which could further alleviate side effects.

The amphetamine conjugates listed above of the present technology demonstrate that by using homoarginine amphetamine conjugate, a shift in the Tₘₐₓ occurs while still retaining AUC and potential clinical effect. By using homoarginine, we are able to demonstrate that hArg-Amp show little release via the IN (intranasal) or IV (intravenous) route yet still maintain a similar AUC.

### Example 9

### Pharmacokinetic Study of hArg-Amp-2HCl and Lys-Amp

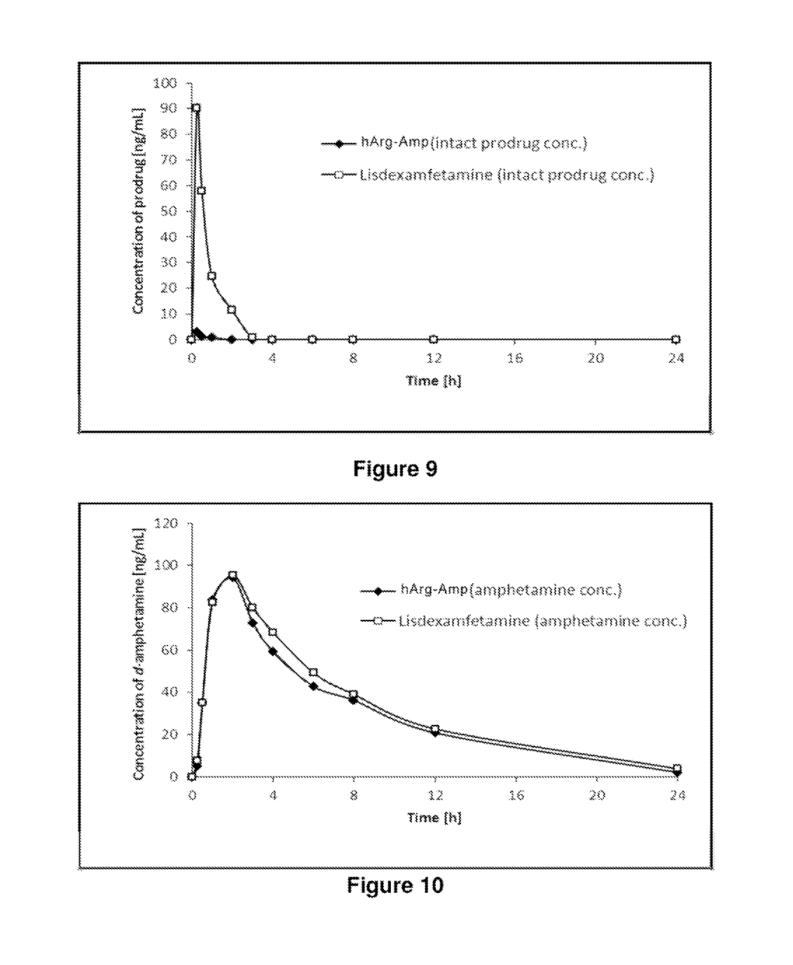

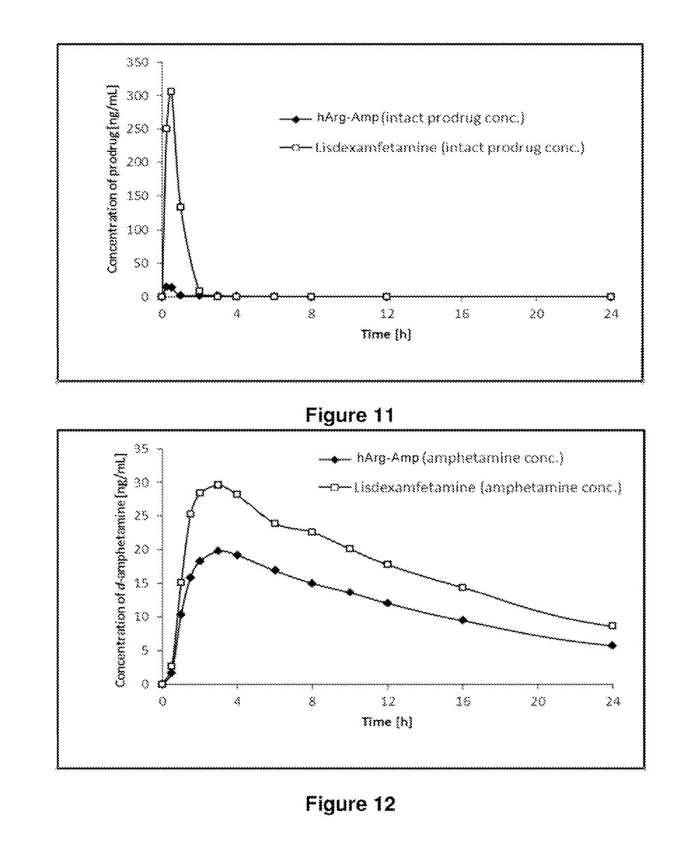

Oral solutions of hArg-Amp-2HCl and Lys-Amp were administered in rats at equimolar doses (4.20 mg/kg and 5.05 mg/kg, respectively). The resulting PK curves of d-amphetamine released from the prodrugs and of intact prodrugs are shown in FIGS. 8 and 9, respectively. hArg-Amp-2HCl and Lys-Amp were also administered orally in dogs at equimolar doses (1.5 mg/kg and 1.8 mg/kg, respectively). The respective PK curves for d-amphetamine and intact prodrugs are shown in FIGS. 10 and 11. The PK parameters for the rat and dog studies are summarized in Tables 6 and 7, respectively.

TABLE 6
PK parameters for hArg-Amp-2HCl
and Lys-Amp after oral administration in rats.

| | d-amphetamine | | | intact prodrug | | |
|---|---|---|---|---|---|---|
| | hArg- | | hArg-Amp- | hArg- | | hArg-Amp- |
| | Amp- | Lys- | 2HCl/ | Amp- | Lys- | 2HCl/ |
| Parameter | 2HCl | Amp | Lys-Amp | 2HCl | Amp | Lys-Amp |
| AUC_{0-24 h} | 447.2 | 596.4 | 75.0% | 1.8 | 74.9 | 2.1%^{†} |
| [ng/mL × h] | | | | | | |
| Cₘₐₓ [ng/mL] | 43.2 | 71.5 | 60.4% | 3.1 | 90.2 | 3.0%^{†} |
| Tₘₐₓ [h] | 3.0 | 2.0 | 150.0% | 0.25 | 0.25 | 100.0% |

^{†}molar ratio

TABLE 7
PK parameters for hArg-Amp-2HCl
and Lys-Amp after oral administration in dogs.

| | d-amphetamine | | | intact prodrug | | |
|---|---|---|---|---|---|---|
| | hArg- | | hArg-Amp- | hArg- | | hArg-Amp- |
| | Amp- | Lys- | 2HCl/ | Amp- | Lys- | 2HCl/ |
| Parameter | 2HCl | Amp | Lys-Amp | 2HCl | Amp | Lys-Amp^{†} |
| AUC_{0-24 h} | 706.2 | 775.0 | 91.1% | 17.0 | 285.8 | 5.1%^{†} |
| [ng/mL × h] | | | | | | |
| Cₘₐₓ [ng/mL] | 94.3 | 95.4 | 98.8% | 14.8 | 305.5 | 4.2%^{†} |
| Tₘₐₓ [h] | 2.0 | 2.0 | 100.0% | 0.25 | 0.5 | 50.0% |

^{†}molar ratio

In rats, hArg-Amp-2HCl released d-amphetamine in an attenuated fashion when compared to Lys-Amp. The plasma concentrations of d-amphetamine released from hArg-Amp-2HCl increased more slowly up to the tₘₐₓ and also decreased more slowly after the tₘₐₓ compared to Lys-Amp. The overall systemic exposure (AUC) to d-amphetamine was reduced by 25% for hArg-Amp-2HCl compared to Lys-Amp. The Cₘₐₓ of d-amphetamine released from hArg-Amp-2HCl was approximately 60% of the Cₘₐₓ for Lys-Amp. Additionally, peak plasma concentrations of d-amphetamine released from hArg-Amp-2HCl were reached one hour later when compared to Lys-Amp. Moreover, intact prodrug concentrations were significantly lower for hArg-Amp-2HCl than for Lys-Amp (Cₘₐₓ values were 3.1 ng/mL and 90.2 ng/mL and AUC values were 1.8 ng/mL×h and 74.9 ng/mL×h for hArg-Amp-2HCl and Lys-Amp, respectively).

In dogs, hArg-Amp-2HCl and Lys-Amp were bioequivalent based on released d-amphetamine. Consequently, the slow onset of d-amphetamine plasma concentrations with hArg-Amp-2HCl in rats was not observed in dogs. Dog plasma concentrations of intact prodrug, however, were considerably lower for hArg-Amp-2HCl than for Lys-Amp, similar to the results found in rats.

### Example 10

### Pharmacokinetic Study of hArg-Amp-2HCl and Lys-Amp in Humans

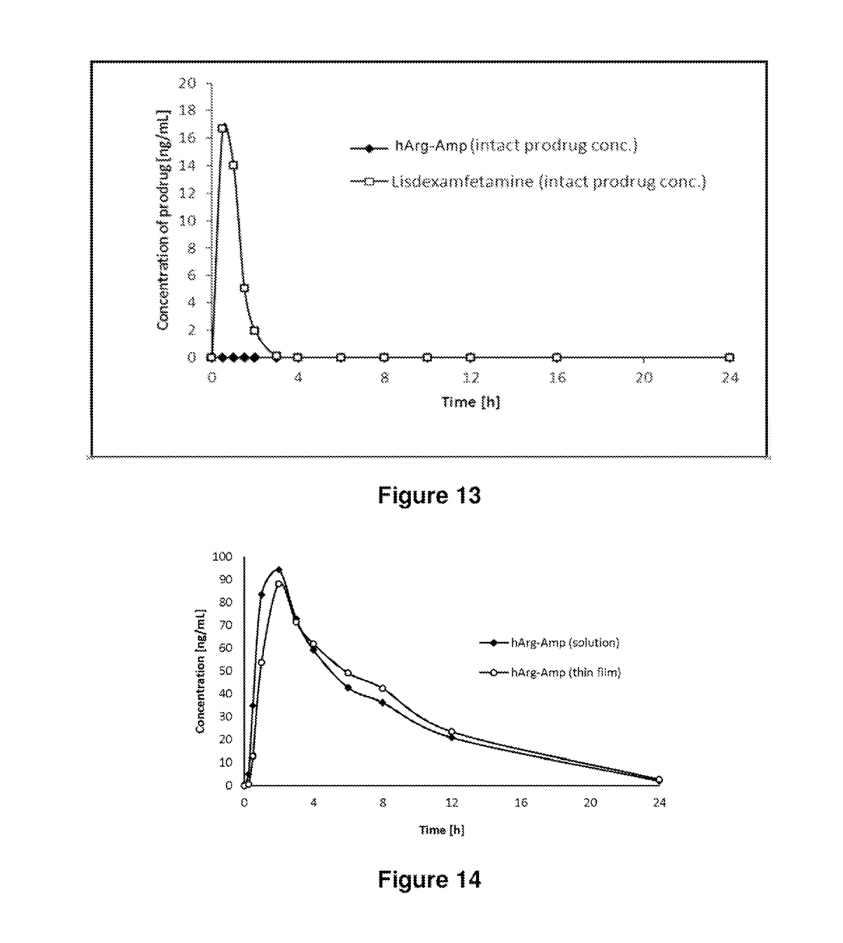

A single-dose, 2-period, 2-treatment, 2-sequence crossover study was conducted comparing hArg-Amp-2HCl 25 mg solution to Vyvanse® 30 mg solution (d-amphetamine equivalent dosages) administered orally in twenty four healthy volunteers under fastened conditions. The resulting PK curves and PK parameters are summarized in FIGS. 12 and 13 and Table 8.

TABLE 8
PK parameters for hArg-Amp-2HCl
and Lys-Amp after oral administration in humans.

| | d-amphetamine | | | intact prodrug | | |
|---|---|---|---|---|---|---|
| | hArg- | | hArg-Amp- | hArg- | | hArg-Amp- |
| | Amp- | Lys- | 2HCl/ | Amp- | Lys- | 2HCl/ |
| Parameter | 2HCl | Amp | Lys-Amp | 2HCl | Amp | Lys-Amp |
| AUC_{0-24 h} | 282.7 | 421.9 | 67.0% | 0 | 19.4 | 0% |
| [ng/mL × h] | | | | | | |
| Cₘₐₓ [ng/mL] | 20.7 | 30.0 | 69.0% | 0 | 18.1 | 0% |
| Tₘₐₓ [h] | 3.15 | 2.85 | 110.5% | 0 | 0.61 | 0% |

In humans, the release of d-amphetamine from hArg-Amp-2HCl was attenuated compared to Lys-Amp. However, the initial increase in d-amphetamine plasma concentrations produced by hArg-Amp-2HCl over the time points before the tₘₐₓ was faster and more similar to Lys-Amp in human subjects than in rats. Both the Cₘₐₓ and AUC value for d-amphetamine released from hArg-Amp-2HCl in humans were approximately two thirds of the respective values for Lys-Amp. These values more closely resemble the data observed in rats. But in disagreement with the rat data, the tₘₐₓ values for d-amphetamine released from hArg-Amp-2HCl and Lys-Amp were very similar in humans (3.15 hours for hArg-Amp-2HCl and 2.85 hours for Lys-Amp) while peak plasma concentrations of d-amphetamine in rats occurred 1 hour later for hArg-Amp-2HCl when compared to Lys-Amp. That is, the shift in peak plasma concentrations of d-amphetamine between hArg-Amp-2HCl and Lys-Amp in rats unexpectedly was not observed in humans.

In addition, no intact prodrug was detected in plasma after oral administration of hArg-Amp-2HCl 25 mg in humans (i.e., all plasma concentrations were below the lower limit of quantitation of 1.00 ng/mL). Plasma concentrations of intact Lys-Amp, however, were still significant and detectable in humans at the same molar dose level. This result is surprising and contrary to the data observed in animals.

### Example 11

### Oral PK of l-homoarginine-d-amphetamine (hArg-Amp) HCl in Dogs

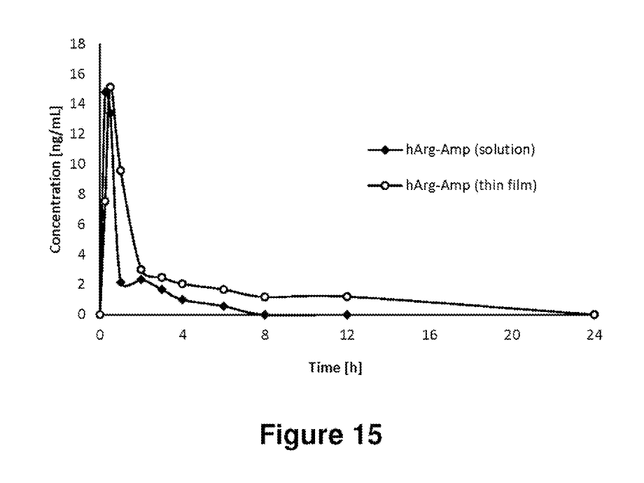

An oral solution and an oral thin film (OTF) of hArg-Amp were administered in dogs at 1.5 mg/kg of amphetamine. The resulting PK curves of d-amphetamine released from the prodrugs and of intact prodrugs for both dosage forms are shown in FIGS. 14 and 15, respectively. In either dosage form, hArg-Amp released similar amounts of d-amphetamine over a 24 hour time period. Both dosage forms were bioequivalent based on released amphetamine.

The homoarginine amphetamine prodrug of the present technology is chemically stable to in vitro hydrolysis of the amide linkage to prevent tampering or removing the amphetamine prior to oral ingestion. Also, the controlled release of amphetamine through oral administration of the homoarginine-amphetamine prodrug of the present technology is an inherent property of the molecule, not related to the formulation. Therefore, the prodrug of the present technology can be easily formulated into different dosage forms. As can be seen from a comparison of the data from the rat, dog and human studies, the absorption of the intact prodrug is not a predictive property of the prodrug. Plasma concentrations of the intact homoarginine-amphetamine prodrug were detected for both rats and dogs, but were below the detectable limit in humans. This result is surprising, especially since the intact prodrug Lys-Amp was detected in the plasma samples of rats, dogs and humans.

The invention is now described in such full, clear, concise and exact terms as to enable any person skilled in the art to which it pertains, to practice the same. It is to be understood that the foregoing describes preferred embodiments of the invention and that modifications may be made therein without departing from the spirit or scope of the invention as set forth in the appended claims.

What is claimed is:

1. A method of reducing or preventing amphetamine or amphetamine derivative side effects in a human comprising the step of orally administering at least one thin film or strip comprising a dose of at least one homoarginine amphetamine dihydrochloride prodrug or homoarginine amphetamine derivative dihydrochloride prodrug, or a combination thereof, wherein the dose is equivalent to amphetamine freebase in the range of about 5 mg to about 40 mg, and wherein the prodrug is below the limit of quantitation in the bloodstream of the human following the oral administration step.

2. The method of claim 1, wherein the prodrug is below the level of 1.00 ng/mL in the bloodstream of the human following the oral administration step.

3. The method of claim 1, wherein the dose is equivalent to amphetamine freebase in the range of about 9 mg to about 30 mg.

4. A composition for reducing or preventing amphetamine or amphetamine derivative side effects in a human, the composition comprising at least one orally administered homoarginine-amphetamine prodrug or salt thereof, or at least one homoarginine-amphetamine derivative prodrug or a salt thereof, wherein either of the prodrugs or salts thereof are present in the composition in an amount equivalent to amphetamine freebase in the range of about 5 mg to about 40 mg, and wherein either of the prodrugs or salts thereof are below the limit of quantitation in the bloodstream of the human following oral administration.

5. The composition of claim 4, wherein either of the prodrugs or salts thereof are below the level of 1.00 ng/mL in the bloodstream of the human following oral administration.

6. The composition of claim 4, wherein either of the prodrugs or salts thereof are provided in a thin film or strip dosage form.

7. The composition of claim 4, wherein the homoarginine-amphetamine prodrug is homoarginine amphetamine dihydrochloride.

8. The composition of claim 4, wherein the homoarginine-amphetamine derivative prodrug is homoarginine amphetamine derivative dihydrochloride.

9. The composition of claim 4, wherein the amount of the prodrug or salt thereof is equivalent to amphetamine freebase in the range of about 9 mg to about 30 mg.

10. A method of reducing or preventing amphetamine or amphetamine derivative side effects in a human comprising the step of orally administering to a human at least one thin film or strip comprising a dose of at least one homoarginine-amphetamine conjugate or salt thereof, or at least one homoarginine-amphetamine derivative conjugate or a salt thereof, wherein the dose is equivalent to amphetamine freebase in the range of about 5 mg to about 40 mg, and wherein the conjugate is below the limit of quantitation in the bloodstream of the human following the oral administration step.

11. A composition for reducing or preventing amphetamine or amphetamine derivative side effects in a human, the composition comprising at least one orally administered homoarginine-amphetamine conjugate or salt thereof, or at least one homoarginine-amphetamine derivative conjugate or a salt thereof, wherein either of the conjugates or salts thereof are present in the composition in an amount equivalent to amphetamine freebase in the range of about 5 mg to about 40 mg, and wherein either of the conjugates or salts thereof are below the limit of quantitation in the bloodstream of the human following oral administration.

12. The composition of claim 11, wherein either of the conjugates or salts thereof are below the level of 1.00 ng/mL in the bloodstream of the human following oral administration.

13. The composition of claim 11, wherein either of the conjugates or salts thereof are provided in a thin film or strip dosage form.

14. The composition of claim 11, wherein the homoarginine-amphetamine conjugate is homoarginine amphetamine dihydrochloride.

15. The composition of claim 11, wherein the homoarginine-amphetamine derivative conjugate is homoarginine amphetamine derivative dihydrochloride.

16. The composition of claim 11, wherein the amount of the conjugate or salt thereof is equivalent to amphetamine freebase in the range of about 9 mg to about 30 mg.

17. A method of reducing or preventing ADD, ADHD, or CNS diseases or disorders in a human comprising the step of orally administering at least one thin film or strip comprising a dose of at least one homoarginine amphetamine dihydrochloride prodrug or homoarginine amphetamine derivative dihydrochloride prodrug, or a combination thereof, wherein the dose is equivalent to amphetamine freebase in the range of about 5 mg to about 40 mg, and wherein the prodrug is below the limit of quantitation in the bloodstream of the human following the oral administration step.

18. A composition for reducing or preventing ADD, ADHD, or negative CNS stimulation side effects, or psychostimulant side effects, diseases or disorders in a human, the composition comprising at least one orally administered homoarginine-amphetamine prodrug or salt thereof, or at least one homoarginine-amphetamine derivative prodrug or a salt thereof, wherein either of the prodrugs or salts thereof are present in the composition in an amount equivalent to amphetamine freebase in the range of about 5 mg to about 40 mg, and wherein either of the prodrugs or salts thereof are below the limit of quantitation in the bloodstream of the human following oral administration.

19. A method of reducing or preventing ADD, ADHD, or negative CNS side effects in a human comprising the step of orally administering to a human at least one thin film or strip comprising a dose of at least one homoarginine-amphetamine conjugate or salt thereof, or at least one homoarginine-amphetamine derivative conjugate or a salt thereof, wherein the dose is equivalent to amphetamine freebase in the range of about 5 mg to about 40 mg, and wherein the conjugate is below the limit of quantitation in the bloodstream of the human following the oral administration step.

20. A composition for reducing or preventing stimulant side effects in a human, the composition comprising at least one orally administered homoarginine-amphetamine conjugate or salt thereof, or at least one homoarginine-amphetamine derivative conjugate or a salt thereof, wherein either of the conjugates or salts thereof are present in the composition in an amount equivalent to amphetamine freebase in the range of about 5 mg to about 40 mg, and wherein either of the conjugates or salts thereof are below the limit of quantitation in the bloodstream of the human following oral administration.
